SEQUENCE LISTING (22 sequences)

SEQ ID NO: 1 - DNA, 1986 bp, Artificial Sequence
  Synthetic
atgacgtcgtcaaagaagtttacagttgaagttgaaccggcgattccggccaaggatggaaaaccgtcggctggaccggtttaccgtagtatctttgctaaagacggttttccagctcatattgacggtttagattcatgttgggatattttccgcctatctgtggagaaataccccaataatcgaatgcttggcacccgtgaatttgtgaatggaaagcatggaccatatgtatggtcgacttacaaacaagtatacgacaaggtgataaaggttggaaatgctatccgtgcgtgtggtgtcgagccaggtggtcggtgtgggatctatggtgccaattgtgcagaatggattatgagcatggaggcatgtaatgctcatgggctttactgtgtacctttatacgataccttaggtgctggtgcaattgaattcattctttgccatgccgaggttacaattgcttttgtagaagagaaaaagatccctgagttgttgaaaacatttccgaaagctggagaatttctgaaaacaattgtgagctttggaaaagttactcctgaacaaagagaacaagctgaaaactttggtttaaaaatacattcatgggatgaattcttgacattgggtgatgataaaaactttgacctgccactgaaggaaaaaactgatatctgtacaataatgtacactagtggaacaactggtgatcctaagggtgttctgatttcaaataacagcatggcaacacttatagctggcgtcaatcgtctactagatagtgcaaaagaatctttgaatcaacatgatgtctatctctcgtttttacctctggcacatatatttgaccgtgtgattgaagaatgttttatcaatcatggagcatctataggattctggcgtggggatgttaaattgctgattgaagacataggggagctgaaacctactattttctgcgctgttcctcgagtgttggataggatttattcaggtttgcaacagaaaatttctgcggggggttttatcaaacgtaacttatttaatctagcctattcatacaaattacgtaatatgaagggagggaaaacacattcagaggcatctccattgagtgacaaaatcgtcttcagtaaggttaagcagggcctaggaggaaatgtacgaattattctatctggagctgctccactagctccacatgtagaagcttacctgaaagtagtggcatgtagtcacgtcctgcaaggatatggcctgacagaaacttgtgctggatcatttgtctcactgccaaacgaaatggagatgctgggtacagtgggcccacctgtaccagttttggatgcccgactggagtctgttccggagatgaactatgatgcttgttcaagcaaaccacaaggagaaatatgtattagaggggatgttctgttttcaggatactacaagcgtgaggaccttacaaaagaagtctttgttgatgggtggttccatacaggtgatatcggtgagtggcaaccagatggaagcatgaaaattattgaccgaaagaaaaacatttttaagctctcacaaggagagtacgtcgcagttgaaaatctggagaatgtttatggaaatgtttctgacattgacacgatatggatatatgggaacagcttcgagttttgtcttgttgctgtggtcaacccaaatgagccagcaatcaaacgttatgctgaagcaaataatatttctggggattttgattcattatgtgaaaatcccaaaattaaagaatacatactcggagagctcgctagaattggaaaagagaaaaagttaaaaggttttgaattcgtcaaagctgttcaccttgaccctgtccctttcgacatggaacgtgaccttctgaccccaacattcaagaagaaaaggccccagatgcttaagtactaccaggatgtaattgataacatgtacaagactattaacaagaagtga

SEQ ID NO: 2 - DNA, 1650 bp, Artificial Sequence
  Synthetic
atggatgcattgaggaagcctaattctgcgaattcaagccctttaactcctatcggattccttgaaagggcagccgtcgtatttgccaactctccttcgatcgtatacaacaatctcatctacacttggagcgatacttttcatcgttgtctacgattagcttcatccatctctcgtctcgctatacgaaaaggcgacgttgtttcagtactcgcaccaaacatccctgccatttatgagcttcattttggcatcactatgactggggccataatcaacaccatcaatacccgtttggatgcgcgtactatctcaatactcctttgtcacagtgaatccaagctcgtctttgttgattaccagttgactcgtcttatacgagaagcggtttctttgatgccagatgcttgtgttcccccacaactcgtcctcatcgtagatgacggacataatctatctttactttctgatcaatttatcaatacttatgaagctatggttgaaacaggggatcctgggttcaattgggttcgtccagatagcgattgggaccctctaacgttgaattacacttctgggacgacttcttcccccaaaggtgttgttaacagccaccgtggatcgttcatagtagcgtttgattctttactggagtggcacgtaccgaaacagccgatcatgctgtggactctaccaatgttccacgcaaatgggtggagcttcgtttggggtatggcagctgttggtggcaccaatgtttgccttcgtaaattcgatgctactattatttatgacaccattcgtaaccaccatgtgacgcacatgtgtggcgcccctgttgtactcaacatgttatcagaaggtaagccacttgaacacacggttcacataatgacagcaggagcaccacctccagcggccgttttgttgcgaaccgagtcgctagggtttgaggtgactcatgggttcgggatgacagaaacaggcgggttagttgtgtcatgctcatggaagaaagaatggaatcgtctgcccgtgactgagaaagcgagattgaaagcgagacaaggagttagaacacttgggatgacggaagtggatattgtggatcccgagtcaggagtaagtgtgactcgagacgggttaactcagggggaattagtgttgcgaggtgggtctattatgttgggttacttaaaagatccggaaacaacaaataaatccgttaaaaacgggtggttttataccggcgacgtggcggtgatgcatccagatggatatctggaaataaaagatagatcaaaagatgtaataataagtggtggtgagaatataagtagtgtggaggttgagtcaatcttgtatcagcatcctgcgattaacgaggccgcggtggtgggacggcctgatgagttttggggcgagtcgccgtgtgctttcgtgagtttgaaagatgataacgggaaggtggctgtgccaacagcggatgagataatgaagttttgtaaaggaaagttgccgggttacatggtacccaaatcggttgtgtttaagaaggatcttccgaagacatctaccggtaagattcagaaatatgtgcttagaaaacttgctaaagatttgggttttgctgtaaaaagtcgaatttag

SEQ ID NO: 3 - DNA, 1440 bp, Artificial Sequence
  Synthetic
atgaccgaagaggaaaaaaataaagcagagtccatggggataaaaacgtatgcatggagcgacttccttcatctggggagtaaaaatccttcagaactgcaaacgcctaaagcaactgatatatgtacaatcatgtacactagtggcactagtggagacccaaaaggtgttatattgacacatgaaaatgctacaacaaacatacgaggggttgatcttttcatggaacaattcgaggacaagatgaccgtggatgacgtttatatatctttcttgcctcttgctcacattcttgatcgtatgattgaagaatactttttccgtagtggtgcctctgtcggcttctatcatggggatatcaatgcgttgaaggaggatttggcagagctaaagcctacttttttggctggagtacctcgagttttggaaaagattcacgaaggtgtgcttaaaggactagaagaagttaatccaaggagaaggaaaatatttagcattttatacaatcacaaactaaaatacatgaaagcaggttacaagcataaatatgcatcaccacttgcagatctgcttgcttttagaaaggttaagaacaggcttggtgggcgaattcgtcttatggtatctggaggagctccgttaagcactgagattgaagagttcatgagggttacttcatgtgcttttgtggcgcaaggatatggtttgacggaaacatgtggtttggctactttaggatttccagatgagatgtgcatgattggaacagttggttcgcccttcgtgtatacagaattacgcctcgaagaagtttcagatatgggctatgacccgttggccaatccaccacgtggtgaaatatgtgttaagggaaaaacgcctttcgcaggttactacaagaatccagaactcactaatgaggtcatgaaagatgggtggtttcatacaggtgacataggagagatgcaaccaaacggggtattgaaaatcatcgacagaaagaaacatctgataaaactatctcaaggggagtatatcgcgcttgaatatctagagaaagtttactgcatcactcccattcttgaagacatctgggtatatggggatagcttcaagtcatcattggtcgcggtagctgtaccaaacaaagaaaacgcagaaaagtgggccgatcaaaagggccttaaagtttcttactctgagctctgcacactaacacagttcagagattatatccaatctgaactgaaatctaccgcggagagaaacaagctaagaggttttgagcatataaaggctataattgtggagccacggacgtttgaaggagaccaggaattgttgactgcaacaatgaagaaacgtagaaataaactgcttaaccgttacaaggaggggatcgacaacctttacaagaacttggctgcaaacaaacgctga

SEQ ID NO: 4 - DNA, 2181 bp, Artificial Sequence
  Synthetic
atggtgtacaagtctttgaattcaatatccatatcagatatagtaaatcttggtatatcacctgaaactgcaactcaacttcatcagaaactaactgaaatcattcagatttatggttttgatgctcctcaaacatggacccagatatccacccggattcttcatccggaccttcccttttgttttcatcagatgatgtattatggatgctatgttgattttggaccggatcctcctgcttggtcacccgacccgaaggatgcaaagttaacaaacataggtagtttattagagagacgcggaaaggagttcttggggcctagttataaagatcccatttcaagctactctgctcttcaggaattttcagccttaaatctagaggtgttttggaaaacaatattggatgaaatgaatataacattttctgtgcctccaaaacgcatattagttgatgacctgtctaaagaaagccagttattgcatccaggtggtcgatggcttcccggagcttatgtaaatccagctagaaattgtttgagtttaagtagcaagagaaggttaagtgatatagcagttatatggcgtgatgaaggaaatgatgatatgccggtcaacaaaatgacgtttcagcagttgcgctcagaggtttggttagttgcatatgcacttgatacattgggagtggaaaaaggatctgcaattgcaatcgatatgcctatggatgtcaaatctgtggtgatttatctagccattgttttagcaggctatgtggttgtatctattgcagatagttttgctgctggtgaaatttcgaccagacttgtattatcaaaagcaaaagcaatttttactcaggatttgatcattcgtggtgacagaagccatcccttgtacagccgagttgttgatgctcaatcacctctagcaattgtcattcctacgagaggctcaagttttagtataaaattacgtgacggtgatatttcttggcatgattttctggaacgagctaacacttacaggaatgttgagtttgttgctgttgaacgacccgttgaagctttctcaaatatccttttctcatcaggaactacaggggaaccgaaggcaattccatggacccttgcaacacctttcaaggctggtgcagacgcttggtgccacatggatgtccacaaaggtgatgttgttgcatggcctactaatcttggatggatgatgggtccttggctaatatatgcttcattgttaaatgggggctcacttgcattatacaacggatctcccctgacttctggatttgccaagtttgttcaggatgcaaaagtaacattgttgggagtgataccaagtattgtgagggcatggagaacaaacaatagtacagccggctttgactggtcaaccatccggtgctttggatcgaccggtgaggcctctaatactgatgaatgtctttggctgatgggaagagctcattacaaaccggtcatcgagtattgcggtggcacagagattggtggtggttttattacaggatctttactgcagcctcagtgtttgtctgctttcagcacaccaagtttgggttgtaaactgttaattcttggcgaagatggaatccctataccacaaaacgctcctggaattggtgaattggctctgaatcccctcatgtttggggcatcgagcacactactaaatgcaaaccactatgatgtctactttaaaggcatgccctcttggaatggtaaggttctaagaaggcatggagatgtatttgagcgcacgtctaaaggatactatcgtgcccatggtcgtgcagatgatactatgaatcttgggggtattaaggtaagttcggttgagattgaacgtgtatgcaactcgattgatgacagaattctcgagacagcggctataggggttacaccttctggtggcgggccagagaggttggtaattgttgttgcttttaaagatggcagtggttcgaaacccgacttaatcaagttgaaggtcacactgaattcagctttacaaaagaatctgaaccctttgtttaaggtttctgatgtggtgccctttccatcacttcctaggacagcaacaaacaaggtaatgagaagggttttgcgacagcagttgactcaaattggtcaaaatagcaagctataa

SEQ ID NO: 5 - DNA, 2175 bp, Artificial Sequence
  Synthetic
atgggtgattcagagggaagcagcattagtactcctacaactgaacaagttggtttcttgtcaaatatcatggaagacaaatcttatagtgctgcagttgcaattatggttgccattgctgtaccgttggttctttcttcagtgtttgcagcgaagaagaaagtgaaacaacgaggcgttcccgttcaagttggtggtgagccaggttttgccatgcgtaactctagatcaaacaaattagttgatgtcccatgggaaggagctagaacaatggctgctctttttgagcagtcttgtaagaagcattcacagcttcggtttcttggtacaaggaagttgattgaaagaagctttgtgagtggtagtgatgggagaaaattcgagaagttacatcttggggagtatcagtgggagacatatgggcagatatttgaacgtgtttgcaactttgcatctggacttattcagcttggtcatgaccctgatactcgtattgccatcttttctgacacacgagctgaatggttaattgcatttgagggatgcttcaggcagaacatcactgtggttaccatatatgcatcattaggtgatgatgccctcattcactctcttaacgagactaaagtatcgaccttgatttgtgattccaaactattgaaaaaagtggctgcagttagttcaagcctgaaaactgtagaaaacttcatctactttgaaagtgacaacactgaagctttaaatgaaatcggtgattggaaaatatcttctttttctgaagtcgagagcttgggacagaagagtccagtaagtgctagactgcctatcaagaaagacgttgcagtgatcatgtatacaagtggcagcacaggtttaccaaagggggtgatgatgactcatgggaatgtagtagcaactgcagctgcggttatgactgtaatcccaaatattgggaccaatgatgtttatctggcatacttaccattggctcatattttcgagttggctgctgagactgtgatggtaactgcaggtattccaattggttatggttcagcactcactttaacagacacatcaaataaaatcaagaaaggaaccttgggagatgcatccatcttgaagccaacgttaatggcagctgttccagctattttagatcgtgtccgagatggagtattaaagaaggttgaggaaaagggaggtttgacaacaaaaatattcaatatagcctacaaaaggcgtttgctagcagtagatggaagttggctgggtgcatgggggttagagaagctattgtgggatgccattgtttttaagaagattcgttctgtacttggaggagatatccgtttcatgctctgtggtggtgctcctttagctgcagatactcagcgatttataaatgtctgcgttggggctccaattggacaaggatatgggctgaccgaaacatgcgctggagctgctttctctgaggcagatgataattctgttgggcgtgttggtccaccacttccttgtgtctatattaaacttgtttcatgggatgaaggtgggtatttaacatcagacaaaccaatgccgcgaggcgaagttgtagttggtgggtacagtgtaaccgctggttactttaataatgaggaaaagaccaatgaggtttacaaggttgatgaaagtgggatgcgttggttctacactggggacattggaaggtttcatcctgatggatgccttgaaatcattgacaggaagaaggatattgtaaaacttcaacatggagagtacatctccttggggaaggttgaggcagcacttgcgtcaagcaagtatgtagagaatgtaatgttacatgccgaccccttccacacttattgtgtcgccttagttgtccctgcgcgtcaggttatagaacagtgggctcaagatgcgggtattagttaccaagattttgctgagttgtgtgataaaaaggaaactgtctctgaggttcagcaatcccttaccaaggtagcaaaagatgcaaaactagacaagtttgaaacgcctgcaaagataaagctgatgccagatccatggactcctgaatctggattagtaacagcggctcttaagttaaaaagggaacaactgaagtccaaatttaaggatgatctggataagctatatgggtga

SEQ ID NO: 6 - DNA, 1980 bp, Artificial Sequence
  Synthetic
atgtcggtttacaccgttaaagtcgaggattcacgggcagcttccggagaaaccccgtcagcagggccggtttacaggtgcatttatgccaaggatgctctcatggaactgccccccggttatgaatctccctgggacttctttagtgagtctgttaaaagaaacccaaagaacccagcactaggtcgtcgtcaagtcatcgatggaaaggctggtggttattcatggctttcatatcaagaagcctacaattctgctctacgcattgcttctgccatcagaagccgatctgttaatcctggggatcggtgtggtatatatggacctaactgtcctgaatggataatctcaatggaggcttgtaacagcaatggcataacctatgttcccctatatgatacacttggtgctaatgcggttgaatacatcatcaaccatgcagaaatttctttagtttttgttcaagagaacaagttgtctgctattttatcatgtcttccaaattgctcatcaaatcttaaaacaatcgtcagctttgggaagttctctgaatcacaaaagaacgaagccatggaacatggcgtcgattgcttctcttgggaagagttttcttcgatggggaatttggaagatgaacttcctgcaaaaaataagactgacatttgcaccataatgtatacaagtggaacaacgggagagcctaagggtgtcgtactaagtaacagagctttcatgtccgaagtcttgtctatgcatgaactactcatagaaacagacaaaccgggcacagaagaagatacctacttctcttttcttcctttggcacatatatttgatcaaataatggagacgtatttcatctacagtggtgcttcgatagggttttggcaaggagatatcagatacttgattgaagaccttcttgtgttgcagccaaccatattttgtggtgttccaagagtttatgaccgcatttatacgggcataatggctaagatttcaactggaggtgctattcggaaggcattatttgattttgcatacaactataaattaaggaaccttgaaaagggaatacaacaagacaaatcagctcctcttttggacaagctggtcttcgataagattaaacaagggtttggaggaagggttcgtcttatgttatctggagccgcacctttgccaaaacacgtggaggaatttttaagagtgacgtgctgtaccgttctctcacaaggatacggacttactgaaagttgtggtggatgctttacatccattgcgaatgtgtactctatgatcgggactgttggtgtacccatgacaactattgaagcaagacttgagtcagtgccagagatgggatatgatgcactcagtagtgtgccatgtggcgaaatttgcctcaggggaaacacactattttctgggtaccacaaacgagacgatctaactgatgctgtccttgtagatggctggttccatacaggtgacattggggaatggcaggcagatggagcaatgaaaatcattgacaggaaaaagaatatattcaaattgtctcaaggagaatatgttgcagttgaaagtattgaaagcacctattcacggtgtcctttggttacctcgatttgggtgtacggcaatagttttgaatcttttctagttgcggttgtggttcccgatagagtagcagttgaagagtttgctgcaaagaacaatgaatcaggagattatgcatcgttgtgcaagaacccaaatgtcaggaaatatgttcttgaagagctgaatgctgaagctcaatgcaataaacttcgcgggtttgagatgctaaaagcagttcatttggatccagtcccatttgacttcgagagggatttaataacaccaacctttaaactaaaaagacagcagcttctaaaatactataaggattgcgttgaacaactatatgctgaagcaaagacatccaagaaatga

SEQ ID NO: 7 - DNA, 1872 bp, Artificial Sequence
  Synthetic
atggaaactcatggaccaaggcttctaggtgcagcttacaaagatcctatcacgagttataaacagttccaaaagttctctgttcaacatctagaggtgtattggtctcttgtgttagaaaagctttcaatccaatttcaggaacgtccaaaatgtatagtagatacttctgacaaatcaaaacacgggggcacatggcttcccggttcagttttgaacattgcggagtgttgtatattgtcaactactgaaacagatgaaaaggttgcgattgtgtggcgggatgaaagatgtgataatctggatgtaaacaagatgacattcaaagaattgcgacaacaagtaatgttggttgcaaatgcattgaagttattgttttcaaaaggagatcctattgcaattgatatgccaatgacagttactgcagtaattctatatttggcgattgtatattctggatttgtggttgtatctatagctgacagttttgcagctaaagagattgcaacacgattacgtgtatctaatgcaaaggctatctttactcaagattacattgttcgaggtggtcgaagatttcctttgtacagtcgagttattgaagccacccaatgtagagccatcgtggttcctgcgataggggaaaacgtagaagttattttaagaaaacaggacatttcatggggcgattttctttctggtgcaaaacagcttcctagcccggattattgctctccagtctatcaatccatagacacgttgacaaacatactcttctcttcgggaacaacaggagacccaaaagctataccatggacgcaaatatctccaatgagatgtgctgctgacggatgggctcatatggatattcaggctggagatgtttattgttggcccacaaatctgggatgggtcatgggacccattgtactttactcgagttttcttaccggtgcaacattggctctttataatggctcccctcttggtcatggttttggaaaatttgttcaggatgcaggagtgacaattttgggcacggttccaagcatagtcaagtcttggaagagtacaagatgtatggaaggactggactggacaaagataaaggcatttgggtcgactggtgaagcttctaatgtcgacgatgacctttggctttcctcaaaggcctactacaaacctgttcttgaatgctgtggaggtaccgagcttgcatcttcttatgttcaagggaatcttctacagccacaagcctttggagcattaagctctgcttcaatgggaaccggatttgtcatatttgacgatcatggagttccttacccggacgatgaaccctgtgttggtgaagtgggtttgtttccagtatatatgggagcatctgatagactactgaatgcagatcatgaaaaaatttacttcaagggaatgccgagttacaaaggaatgcaactaaggagacatggagatatcatcaagagaacaattggaggatatttggttgtacaaggcagggctgatgataccatgaaccttggtggcataaagacgagctcaatagaaattgagcgtgtttgtgaacaagctgatggaagcatcatggaaactgctgcagtcagtgttgcacctgcaaccggtggtccagaactattagccatatttgtggtactaaagaacggttgcaacactcaaccacaggacctaaagatgatattttcaaaggccattcaaaaaaacctcaacccattgttcaaggtgagctttgtaaaggttgttccagagttccctcgaaccgcttctaacaagttattgagaagagttttaaggaatcaagtgaaggaagagcttcaaactcgaagtaaaatataa

SEQ ID NO: 8 - DNA, 2082 bp, Artificial Sequence
  Synthetic
atggagatcactaaaagcatccaagaattaggattacaagatctactaaacactggattaacacctaatgatgcaaaatcactgcaaatcgagattaaacacatcattaatagtcaaactactaattcaaacccagttgagttatggcgtcaaatcacttctgcaaagctgcttaaaccctcttatcctcattcgttgcaccagctcatctactacgcggtgtactgtaactatgatgcatccatctatggtcctcccctgtattggtttccatctgaaattgattctaaaaggtcaaacttggggaacattatggaaactcatggaccaaggcttctaggtgcagcttacaaagatcctatcacgagttataaacagttccaaaagttctctgttcaacatctagaggtgtattggtctcttgtgttagaaaagctttcaatccaatttcaggaacgtccaaaatgtatagtagatacttctgacaaatcaaaacacgggggcacatggcttcccggttcagttttgaacattgcggagtgttgtatattgtcaactagtgaaacagatgataaggttgcgattgtatggcgggatgaaagatgtgataatctggatgtaaacaagatgacattcaaagaattgcgacaacaagtaatgttggttgcaaatgcattgaagttattgttttcaaaaggagatcctattgcaattgatatgccaatgacagttactgcagtaattctatatttggcgattgtatattctggatttgtggttgtatctatagctgacagttttgcagctaaagagattgcaacacgattacgtgtatctaatgcaaaggctatctttactcaagattacattgttcgaggtggtcgaagatttcctttgtacagtcgagttattgaagccacccaatgtagagccatcgtggttcctgcgataggggaaaacgtagaagttattttaagaaaacaggacatttcatggggcgattttctttctggtgcaaaacagcttcctagcccggattattgctctccagtctatcaatccatagacacgttgacaaacatactcttctcttcgggaacaacaggagacccaaaagctataccatggacgcaaatatctccaatgagatgtgctgctgacggatgggctcatatggatattcaggctggagatgtttattgttggcccacaaatctgggatgggtcatgggacccattgtactttactcgagttttcttaccggtgcaacattggctctttataatggctcccctcttggtcatggttttggaaaatttgttcaggatgcaggagtgacaattttgggcacggttccaagcatagtcaagtcttggaagagtacaagatgtatggaaggactggactggacaaagataaaggcatttgggtcgactggtgaagcttctaatgtcgacgatgacctttggctttcctcaaaggcctactacaaacctgttcttgaatgctgtggaggtaccgagcttgcatcttcttatgttcaagggaatcttctacagccacaagcctttggagcattaagctctgcttcaatgggaaccggatttgtcatatttgacgatcatggagttccttacccggacgatgaaccctgtgttggtgaagtgggtttgtttccagtatatatgggagcatctgatagactactgaatgcagatcatgaaaaaatttacttcaagggaatgccgagttacaaaggaatgcaactaaggagacatggagatatcatcaagagaacaattggaggatatttggttgtacaaggcagggctgatgataccatgaaccttggtggcataaagacgagctcaatagaaattgagcgtgtttgtgaacaagctgatggaagcatcatggaaactgctgcagtcagtgttgcacctgcaaccggtggtccagaactattagccatatttgtggtactaaagaacggttgcaacactcaaccacaggacctaaagatgatattttcaaaggccattcaaaaaaacctcaacccattgttcaaggttttctcctaa

SEQ ID NO: 9 - DNA, 2181 bp, Artificial Sequence; identical to SEQ ID NO: 4
  Synthetic

SEQ ID NO: 10 - DNA, 1560 bp, Artificial Sequence
  Synthetic
atgacgtttcagcagttgcgctcagaggtttggttagttgcatatgcacttgatacattgggagtggaaaaaggatctgcaattgcaatcgatatgcctatggatgtcaaatctgtggtgatttatctagccattgttttagcaggctatgtggttgtatctattgcagatagttttgctgctggtgaaatttcgaccagacttgtattatcaaaagcaaaagcaatttttactcaggatttgatcattcgtggtgacagaagccatcccttgtacagccgagttgttgatgctcaatcacctctagcaattgtcattcctacgagaggctcaagttttagtataaaattacgtgacggtgatatttcttggcatgattttctggaacgagctaacacttacaggaatgttgagtttgttgctgttgaacgacccgttgaagctttctcaaatatccttttctcatcaggaactacaggggaaccgaaggcaattccatggacccttgcaacacctttcaaggctggtgcagacgcttggtgccacatggatgtccacaaaggtgatgttgttgcatggcctactaatcttggatggatgatgggtccttggctaatatatgcttcattgttaaatgggggctcacttgcattatacaacggatctcccctgacttctggatttgccaagtttgttcaggatgcaaaagtaacattgttgggagtgataccaagtattgtgagggcatggagaacaaacaatagtacagccggctttgactggtcaaccatccggtgctttggatcgaccggtgaggcctctaatactgatgaatgtctttggctgatgggaagagctcattacaaaccggtcatcgagtattgcggtggcacagagattggtggtggttttattacaggatctttactgcagcctcagtgtttgtctgctttcagcacaccaagtttgggttgtaaactgttaattcttggcgaagatggaatccctataccacaaaacgctcctggaattggtgaattggctctgaatcccctcatgtttggggcatcgagcacactactaaatgcaaaccactatgatgtctactttaaaggcatgccctcttggaatggtaaggttctaagaaggcatggagatgtatttgagcgcacgtctaaaggatactatcgtgcccatggtcgtgcagatgatactatgaatcttgggggtattaaggtaagttcggttgagattgaacgtgtatgcaactcgattgatgacagaattctcgagacagcggctataggggttacaccttctggtggcgggccagagaggttggtaattgttgttgcttttaaagatggcagtggttcgaaacccgacttaatcaagttgaaggtcacactgaattcagctttacaaaagaatctgaaccctttgtttaaggtttctgatgtggtgccctttccatcacttcctaggacagcaacaaacaaggtaatgagaagggttttgcgacagcagttgactcaaattggtcaaaatagcaagctataa

SEQ ID NO: 11 - DNA, 1770 bp, Artificial Sequence
  Synthetic
atgaatataacattttctgtgcctccaaaacgcatattagttgatgacctgtctaaagaaagccagttattgcatccaggtggtcgatggcttcccggagcttatgtaaatccagctagaaattgtttgagtttaagtagcaagagaaggttaagtgatatagcagttatatggcgtgatgaaggaaatgatgatatgccggtcaacaaaatgacgtttcagcagttgcgctcagaggtttggttagttgcatatgcacttgatacattgggagtggaaaaaggatctgcaattgcaatcgatatgcctatggatgtcaaatctgtggtgatttatctagccattgttttagcaggctatgtggttgtatctattgcagatagttttgctgctggtgaaatttcgaccagacttgtattatcaaaagcaaaagcaatttttactcaggatttgatcattcgtggtgacagaagccatcccttgtacagccgagttgttgatgctcaatcacctctagcaattgtcattcctacgagaggctcaagttttagtataaaattacgtgacggtgatatttcttggcatgattttctggaacgagctaacacttacaggaatgttgagtttgttgctgttgaacgacccgttgaagctttctcaaatatccttttctcatcaggaactacaggggaaccgaaggcaattccatggacccttgcaacacctttcaaggctggtgcagacgcttggtgccacatggatgtccacaaaggtgatgttgttgcatggcctactaatcttggatggatgatgggtccttggctaatatatgcttcattgttaaatgggggctcacttgcattatacaacggatctcccctgacttctggatttgccaagtttgttcaggatgcaaaagtaacattgttgggagtgataccaagtattgtgagggcatggagaacaaacaatagtacagccggctttgactggtcaaccatccggtgctttggatcgaccggtgaggcctctaatactgatgaatgtctttggctgatgggaagagctcattacaaaccggtcatcgagtattgcggtggcacagagattggtggtggttttattacaggatctttactgcagcctcagtgtttgtctgctttcagcacaccaagtttgggttgtaaactgttaattcttggcgaagatggaatccctataccacaaaacgctcctggaattggtgaattggctctgaatcccctcatgtttggggcatcgagcacactactaaatgcaaaccactatgatgtctactttaaaggcatgccctcttggaatggtaaggttctaagaaggcatggagatgtatttgagcgcacgtctaaaggatactatcgtgcccatggtcgtgcagatgatactatgaatcttgggggtattaaggtaagttcggttgagattgaacgtgtatgcaactcgattgatgacagaattctcgagacagcggctataggggttacaccttctggtggcgggccagagaggttggtaattgttgttgcttttaaagatggcagtggttcgaaacccgacttaatcaagttgaaggtcacactgaattcagctttacaaaagaatctgaaccctttgtttaaggtttctgatgtggtgccctttccatcacttcctaggacagcaacaaacaaggtaatgagaagggttttgcgacagcagttgactcaaattggtcaaaatagcaagctataa

SEQ ID NO: 12 - protein, 661 aa, Artificial Sequence
  Synthetic
MTSSKKFTVEVEPAIPAKDGKPSAGPVYRSIFAKDGFPAHIDGLDSCWDIFRLSVEKYPNNRMLGTREFVNGKHGPYVWSTYKQVYDKVIKVGNAIRACGVEPGGRCGIYGANCAEWIMSMEACNAHGLYCVPLYDTLGAGAIEFILCHAEVTIAFVEEKKIPELLKTFPKAGEFLKTIVSFGKVTPEQREQAENFGLKIHSWDEFLTLGDDKNFDLPLKEKTDICTIMYTSGTTGDPKGVLISNNSMATLIAGVNRLLDSAKESLNQHDVYLSFLPLAHIFDRVIEECFINHGASIGFWRGDVKLLIEDIGELKPTIFCAVPRVLDRIYSGLQQKISAGGFIKRNLFNLAYSYKLRNMKGGKTHSEASPLSDKIVFSKVKQGLGGNVRIILSGAAPLAPHVEAYLKVVACSHVLQGYGLTETCAGSFVSLPNEMEMLGTVGPPVPVLDARLESVPEMNYDACSSKPQGEICIRGDVLFSGYYKREDLTKEVFVDGWFHTGDIGEWQPDGSMKIIDRKKNIFKLSQGEYVAVENLENVYGNVSDIDTIWIYGNSFEFCLVAVVNPNEPAIKRYAEANNISGDFDSLCENPKIKEYILGELARIGKEKKLKGFEFVKAVHLDPVPFDMERDLLTPTFKKKRPQMLKYYQDVIDNMYKTINKK

SEQ ID NO: 13 - protein, 549 aa, Artificial Sequence
  Synthetic
MDALRKPNSANSSPLTPIGFLERAAVVFANSPSIVYNNLIYTWSDTFHRCLRLASSISRLAIRKGDVVSVLAPNIPAIYELHFGITMTGAIINTINTRLDARTISILLCHSESKLVFVDYQLTRLIREAVSLMPDACVPPQLVLIVDDGHNLSLLSDQFINTYEAMVETGDPGFNWVRPDSDWDPLTLNYTSGTTSSPKGVVNSHRGSFIVAFDSLLEWHVPKQPIMLWTLPMFHANGWSFVWGMAAVGGTNVCLRKFDATIIYDTIRNHHVTHMCGAPVVLNMLSEGKPLEHTVHIMTAGAPPPAAVLLRTESLGFEVTHGFGMTETGGLVVSCSWKKEWNRLPVTEKARLKARQGVRTLGMTEVDIVDPESGVSVTRDGLTQGELVLRGGSIMLGYLKDPETTNKSVKNGWFYTGDVAVMHPDGYLEIKDRSKDVIISGGENISSVEVESILYQHPAINEAAVVGRPDEFWGESPCAFVSLKDDNGKVAVPTADEIMKFCKGKLPGYMVPKSVVFKKDLPKTSTGKIQKYVLRKLAKDLGFAVKSRI

SEQ ID NO: 14 - protein, 479 aa, Artificial Sequence
  Synthetic
MTEEEKNKAESMGIKTYAWSDFLHLGSKNPSELQTPKATDICTIMYTSGTSGDPKGVILTHENATTNIRGVDLFMEQFEDKMTVDDVYISFLPLAHILDRMIEEYFFRSGASVGFYHGDINALKEDLAELKPTFLAGVPRVLEKIHEGVLKGLEEVNPRRRKIFSILYNHKLKYMKAGYKHKYASPLADLLAFRKVKNRLGGRIRLMVSGGAPLSTEIEEFMRVTSCAFVAQGYGLTETCGLATLGFPDEMCMIGTVGSPFVYTELRLEEVSDMGYDPLANPPRGEICVKGKTPFAGYYKNPELTNEVMKDGWFHTGDIGEMQPNGVLKIIDRKKHLIKLSQGEYIALEYLEKVYCITPILEDIWVYGDSFKSSLVAVAVPNKENAEKWADQKGLKVSYSELCTLTQFRDYIQSELKSTAERNKLRGFEHIKAIIVEPRTFEGDQELLTATMKKRRNKLLNRYKEGIDNLYKNLAANKR

SEQ ID NO: 15 - protein, 726 aa, Artificial Sequence
  Synthetic
MVYKSLNSISISDIVNLGISPETATQLHQKLTEIIQIYGFDAPQTWTQISTRILHPDLPFCFHQMMYYGCYVDFGPDPPAWSPDPKDAKLTNIGSLLERRGKEFLGPSYKDPISSYSALQEFSALNLEVFWKTILDEMNITFSVPPKRILVDDLSKESQLLHPGGRWLPGAYVNPARNCLSLSSKRRLSDIAVIWRDEGNDDMPVNKMTFQQLRSEVWLVAYALDTLGVEKGSAIAIDMPMDVKSVVIYLAIVLAGYVVVSIADSFAAGEISTRLVLSKAKAIFTQDLIIRGDRSHPLYSRVVDAQSPLAIVIPTRGSSFSIKLRDGDISWHDFLERANTYRNVEFVAVERPVEAFSNILFSSGTTGEPKAIPWTLATPFKAGADAWCHMDVHKGDVVAWPTNLGWMMGPWLIYASLLNGGSLALYNGSPLTSGFAKFVQDAKVTLLGVIPSIVRAWRTNNSTAGFDWSTIRCFGSTGEASNTDECLWLMGRAHYKPVIEYCGGTEIGGGFITGSLLQPQCLSAFSTPSLGCKLLILGEDGIPIPQNAPGIGELALNPLMFGASSTLLNANHYDVYFKGMPSWNGKVLRRHGDVFERTSKGYYRAHGRADDTMNLGGIKVSSVEIERVCNSIDDRILETAAIGVTPSGGGPERLVIVVAFKDGSGSKPDLIKLKVTLNSALQKNLNPLFKVSDVVPFPSLPRTATNKVMRRVLRQQLTQIGQNSKL

SEQ ID NO: 16 - protein, 724 aa, Artificial Sequence
  Synthetic
MGDSEGSSISTPTTEQVGFLSNIMEDKSYSAAVAIMVAIAVPLVLSSVFAAKKKVKQRGVPVQVGGEPGFAMRNSRSNKLVDVPWEGARTMAALFEQSCKKHSQLRFLGTRKLIERSFVSGSDGRKFEKLHLGEYQWETYGQIFERVCNFASGLIQLGHDPDTRIAIFSDTRAEWLIAFEGCFRQNITVVTIYASLGDDALIHSLNETKVSTLICDSKLLKKVAAVSSSLKTVENFIYFESDNTEALNEIGDWKISSFSEVESLGQKSPVSARLPIKKDVAVIMYTSGSTGLPKGVMMTHGNVVATAAAVMTVIPNIGTNDVYLAYLPLAHIFELAAETVMVTAGIPIGYGSALTLTDTSNKIKKGTLGDASILKPTLMAAVPAILDRVRDGVLKKVEEKGGLTTKIFNIAYKRRLLAVDGSWLGAWGLEKLLWDAIVFKKIRSVLGGDIRFMLCGGAPLAADTQRFINVCVGAPIGQGYGLTETCAGAAFSEADDNSVGRVGPPLPCVYIKLVSWDEGGYLTSDKPMPRGEVVVGGYSVTAGYFNNEEKTNEVYKVDESGMRWFYTGDIGRFHPDGCLEIIDRKKDIVKLQHGEYISLGKVEAALASSKYVENVMLHADPFHTYCVALVVPARQVIEQWAQDAGISYQDFAELCDKKETVSEVQQSLTKVAKDAKLDKFETPAKIKLMPDPWTPESGLVTAALKLKREQLKSKFKDDLDKLYG

SEQ ID NO: 17 - protein, 659 aa, Artificial Sequence
  Synthetic
MSVYTVKVEDSRAASGETPSAGPVYRCIYAKDALMELPPGYESPWDFFSESVKRNPKNPALGRRQVIDGKAGGYSWLSYQEAYNSALRIASAIRSRSVNPGDRCGIYGPNCPEWIISMEACNSNGITYVPLYDTLGANAVEYIINHAEISLVFVQENKLSAILSCLPNCSSNLKTIVSFGKFSESQKNEAMEHGVDCFSWEEFSSMGNLEDELPAKNKTDICTIMYTSGTTGEPKGVVLSNRAFMSEVLSMHELLIETDKPGTEEDTYFSFLPLAHIFDQIMETYFIYSGASIGFWQGDIRYLIEDLLVLQPTIFCGVPRVYDRIYTGIMAKISTGGAIRKALFDFAYNYKLRNLEKGIQQDKSAPLLDKLVFDKIKQGFGGRVRLMLSGAAPLPKHVEEFLRVTCCTVLSQGYGLTESCGGCFTSIANVYSMIGTVGVPMTTIEARLESVPEMGYDALSSVPCGEICLRGNTLFSGYHKRDDLTDAVLVDGWFHTGDIGEWQADGAMKIIDRKKNIFKLSQGEYVAVESIESTYSRCPLVTSIWVYGNSFESFLVAVVVPDRVAVEEFAAKNNESGDYASLCKNPNVRKYVLEELNAEAQCNKLRGFEMLKAVHLDPVPFDFERDLITPTFKLKRQQLLKYYKDCVEQLYAEAKTSKK

SEQ ID NO: 18 - protein, 623 aa, Artificial Sequence
  Synthetic
METHGPRLLGAAYKDPITSYKQFQKFSVQHLEVYWSLVLEKLSIQFQERPKCIVDTSDKSKHGGTWLPGSVLNIAECCILSTTETDEKVAIVWRDERCDNLDVNKMTFKELRQQVMLVANALKLLFSKGDPIAIDMPMTVTAVILYLAIVYSGFVVVSIADSFAAKEIATRLRVSNAKAIFTQDYIVRGGRRFPLYSRVIEATQCRAIVVPAIGENVEVILRKQDISWGDFLSGAKQLPSPDYCSPVYQSIDTLTNILFSSGTTGDPKAIPWTQISPMRCAADGWAHMDIQAGDVYCWPTNLGWVMGPIVLYSSFLTGATLALYNGSPLGHGFGKFVQDAGVTILGTVPSIVKSWKSTRCMEGLDWTKIKAFGSTGEASNVDDDLWLSSKAYYKPVLECCGGTELASSYVQGNLLQPQAFGALSSASMGTGFVIFDDHGVPYPDDEPCVGEVGLFPVYMGASDRLLNADHEKIYFKGMPSYKGMQLRRHGDIIKRTIGGYLVVQGRADDTMNLGGIKTSSIEIERVCEQADGSIMETAAVSVAPATGGPELLAIFVVLKNGCNTQPQDLKMIFSKAIQKNLNPLFKVSFVKVVPEFPRTASNKLLRRVLRNQVKEELQTRSKI

SEQ ID NO: 19 - protein, 693 aa, Artificial Sequence
  Synthetic
MEITKSIQELGLQDLLNTGLTPNDAKSLQIEIKHIINSQTTNSNPVELWRQITSAKLLKPSYPHSLHQLIYYAVYCNYDASIYGPPLYWFPSEIDSKRSNLGNIMETHGPRLLGAAYKDPITSYKQFQKFSVQHLEVYWSLVLEKLSIQFQERPKCIVDTSDKSKHGGTWLPGSVLNIAECCILSTSETDDKVAIVWRDERCDNLDVNKMTFKELRQQVMLVANALKLLFSKGDPIAIDMPMTVTAVILYLAIVYSGFVVVSIADSFAAKEIATRLRVSNAKAIFTQDYIVRGGRRFPLYSRVIEATQCRAIVVPAIGENVEVILRKQDISWGDFLSGAKQLPSPDYCSPVYQSIDTLTNILFSSGTTGDPKAIPWTQISPMRCAADGWAHMDIQAGDVYCWPTNLGWVMGPIVLYSSFLTGATLALYNGSPLGHGFGKFVQDAGVTILGTVPSIVKSWKSTRCMEGLDWTKIKAFGSTGEASNVDDDLWLSSKAYYKPVLECCGGTELASSYVQGNLLQPQAFGALSSASMGTGFVIFDDHGVPYPDDEPCVGEVGLFPVYMGASDRLLNADHEKIYFKGMPSYKGMQLRRHGDIIKRTIGGYLVVQGRADDTMNLGGIKTSSIEIERVCEQADGSIMETAAVSVAPATGGPELLAIFVVLKNGCNTQPQDLKMIFSKAIQKNLNPLFKVFS

SEQ ID NO: 20 - protein, 726 aa, Artificial Sequence; identical to SEQ ID NO: 15
  Synthetic

SEQ ID NO: 21 - protein, 519 aa, Artificial Sequence
  Synthetic
MTFQQLRSEVWLVAYALDTLGVEKGSAIAIDMPMDVKSVVIYLAIVLAGYVVVSIADSFAAGEISTRLVLSKAKAIFTQDLIIRGDRSHPLYSRVVDAQSPLAIVIPTRGSSFSIKLRDGDISWHDFLERANTYRNVEFVAVERPVEAFSNILFSSGTTGEPKAIPWTLATPFKAGADAWCHMDVHKGDVVAWPTNLGWMMGPWLIYASLLNGGSLALYNGSPLTSGFAKFVQDAKVTLLGVIPSIVRAWRTNNSTAGFDWSTIRCFGSTGEASNTDECLWLMGRAHYKPVIEYCGGTEIGGGFITGSLLQPQCLSAFSTPSLGCKLLILGEDGIPIPQNAPGIGELALNPLMFGASSTLLNANHYDVYFKGMPSWNGKVLRRHGDVFERTSKGYYRAHGRADDTMNLGGIKVSSVEIERVCNSIDDRILETAAIGVTPSGGGPERLVIVVAFKDGSGSKPDLIKLKVTLNSALQKNLNPLFKVSDVVPFPSLPRTATNKVMRRVLRQQLTQIGQNSKL

SEQ ID NO: 22 - protein, 589 aa, Artificial Sequence
  Synthetic
MNITFSVPPKRILVDDLSKESQLLHPGGRWLPGAYVNPARNCLSLSSKRRLSDIAVIWRDEGNDDMPVNKMTFQQLRSEVWLVAYALDTLGVEKGSAIAIDMPMDVKSVVIYLAIVLAGYVVVSIADSFAAGEISTRLVLSKAKAIFTQDLIIRGDRSHPLYSRVVDAQSPLAIVIPTRGSSFSIKLRDGDISWHDFLERANTYRNVEFVAVERPVEAFSNILFSSGTTGEPKAIPWTLATPFKAGADAWCHMDVHKGDVVAWPTNLGWMMGPWLIYASLLNGGSLALYNGSPLTSGFAKFVQDAKVTLLGVIPSIVRAWRTNNSTAGFDWSTIRCFGSTGEASNTDECLWLMGRAHYKPVIEYCGGTEIGGGFITGSLLQPQCLSAFSTPSLGCKLLILGEDGIPIPQNAPGIGELALNPLMFGASSTLLNANHYDVYFKGMPSWNGKVLRRHGDVFERTSKGYYRAHGRADDTMNLGGIKVSSVEIERVCNSIDDRILETAAIGVTPSGGGPERLVIVVAFKDGSGSKPDLIKLKVTLNSALQKNLNPLFKVSDVVPFPSLPRTATNKVMRRVLRQQLTQIGQNSKL

(19) United States
(12) Patent Application Publication
(10) Pub. No.: US 2024/0150744 A1
(43) Pub. Date: May 9, 2024

(54) ACYL ACTIVATING ENZYME AND A TRANSGENIC CELL, TISSUE, AND ORGANISM COMPRISING SAME

(71) Applicant: YEDA RESEARCH AND DEVELOPMENT CO. LTD., Rehovot (IL)

(72) Inventors: Asaph AHARONI, Tel Aviv (IL); Prashant SONAWANE, Rehovot (IL); Adam JOZWIAK, Rehovot (IL); Paula BERMAN, Rehovot (IL); Luis DE-HARO, Rehovot (IL)

(21) Appl. No.: 18/281,341

(22) PCT Filed: Mar. 10, 2022
(86) PCT No.: PCT/IL2022/050278

§ 371 Date: Sep. 11, 2023

Related U.S. Application Data
(60) Provisional application No. 63/158,967, filed on Mar. 10, 2021.

(51) Int. Cl. C12N 9/00; C12N 15/82; C12P 17/18

(52) U.S. Cl. CPC C12N 9/93; C12N 15/8243; C12P 17/182

(57) ABSTRACT

The present invention provides polynucleotide sequences derived from Helichrysum umbraculigerum and encoding a protein or a plurality thereof belonging to the acyl activating enzyme (AAE) family. Further provided are an artificial nucleic acid molecule including the polynucleotide, a transgenic cell, tissue, or plant including same. Further provided are method for synthesizing an acyl coenzyme A (CoA).

### CROSS-REFERENCE TO RELATED APPLICATIONS

This application claims the benefit of priority of U.S. Provisional Patent Application No. 63/158,967, titled “ACYL ACTIVATING ENZYME AND A TRANSGENIC CELL, TISSUE, AND ORGANISM COMPRISING SAME”, filed Mar. 10, 2021, the content of which are incorporated herein by reference in their entirety.

### FIELD OF INVENTION

The present invention relates to acyl activating enzymes (AAE) including polynucleotides encoding same, and methods of using same, such as for producing an acyl coenzyme A (CoA).

### BACKGROUND

Cannabinoids are typical of Cannabis sativa L. (Cannabis), although some specific compounds have also been identified in other flowering plants, liverworts, and fungi. One of these plants is Helichrysum umbraculigerum Less (H. umbraculigerum). This perennial South-African plant is the only known plant other than Cannabis, producing cannabigerolic acid (CBGA), the five-carbon alkyl precursor of all the major cannabinoids.

The first enzymatic step in cannabinoid biosynthesis in Cannabis is the formation of olivetolic acid by a polyketide synthase enzyme that catalyzes the condensation of hexanoyl-coenzyme A (CoA) with three molecules of malonyl-CoA. The major cannabinoids, including Δ⁹-tetrahydrocannabinolic acid and cannabidiolic acid, are formed from the precursor hexanoyl-CoA, which is a medium chain fatty acyl-CoA. Other cannabinoids with variant side-chains are formed from aliphatic-CoAs of different lengths (e.g., Δ⁹-tetrahydrocannabivarinic acid is formed from an n-butyryl-CoA primer).

Hexanoyl-CoA and other acyl-CoA thioesters in plants are synthesized by acyl-activating enzymes (AAEs, also called acyl-CoA synthetases) that catalyze the activation of carboxylic acid substrates using ATP. These enzymes act on a variety of carboxylate acids including short-, medium-, long- and very long-chain fatty acids, jasmonate precursors, phenylpropanoid-derived acids (e.g., cinnamic acid) and other organic acids such as malonate, acetate and citrate. Very few medium-chain acyl CoA synthetases have been previously identified in nature. In plants, three enzymes from Arabidopsis thaliana, AAE7, At4g05160 and At5g63380 have been shown to form hexanoyl-CoA from hexanoate.

Cannabinoids are valuable natural products. Genes encoding enzymes involved in cannabinoid biosynthesis will be useful in genetic and/or metabolic engineering of cannabinoids. Such genes may also prove useful for creation, via marker-assisted selection, of specific cannabis varieties for the production of cannabinoid-based pharmaceuticals, or for reconstituting cannabinoid biosynthesis in heterologous organisms such as bacteria or yeast, or for producing cannabinoids in cell-free systems that utilize recombinant proteins.

Genes encoding enzymes of cannabinoid biosynthesis can also be useful in synthesis of cannabinoid analogs and synthesis of analogs of cannabinoid precursors. Cannabinoid analogs have been previously synthesized and may be useful as pharmaceutical products. There remains a need in the art to identify enzymes, and nucleotide sequences encoding such enzymes, that are involved in the synthesis of aromatic polyketides.

### SUMMARY

According to a first aspect, there is provided an isolated DNA molecule comprising a nucleic acid sequence having at least 89% homology to SEQ ID Nos.: 1-11, or any combination thereof.

According to another aspect, there is provided an artificial nucleic acid molecule comprising a nucleic acid sequence having at least 89% homology to any one of SEQ ID Nos.: 1-11, or any combination thereof.

According to another aspect, there is provided a plasmid or an Agrobacterium comprising a nucleic acid sequence having at least 89% homology to any one of SEQ ID Nos.: 1-11, or any combination thereof.

According to another aspect, there is provided an isolated protein encoded by any one of: (a) the isolated DNA molecule of the invention; (b) the artificial vector disclosed herein; and (c) the plasmid or Agrobacterium disclosed herein.

According to another aspect, there is provided a transgenic cell comprising: (a) a nucleic acid sequence having at least 89% homology to any one of SEQ ID Nos.: 1-11, or any combination thereof; (b) the artificial nucleic acid molecule disclosed herein; (c) the plasmid or Agrobacterium disclosed herein; (d) the isolated protein disclosed herein; or (e) any combination of (a) to (d).

According to another aspect, there is provided an extract derived from the herein disclosed transgenic cell, or any fraction thereof.

According to another aspect, there is provided a transgenic plant, a transgenic plant tissue or a plant part, comprising: (a) a nucleic acid sequence having at least 89% homology to any one of SEQ ID Nos.: 1-11, or any combination thereof; (b) the artificial vector disclosed herein; (c) the plasmid or Agrobacterium disclosed herein; (d) the isolated protein disclosed herein; (e) the transgenic cell disclosed herein; or (f) any combination of (a) to (e).

According to another aspect, there is provided a composition comprising: (a) the isolated DNA molecule of the invention; (b) the artificial vector disclosed herein; (c) the plasmid or Agrobacterium disclosed herein; (c) the isolated protein disclosed herein; (d) the transgenic cell disclosed herein; (e) the extract disclosed herein; (f) the transgenic plant tissue or plant part disclosed herein; or (g) any combination of (a) to (g), and an acceptable carrier.

According to another aspect, there is provided a method for synthesizing acyl coenzyme A (CoA) comprising the steps: (a) providing a cell comprising an artificial vector comprising a nucleic acid sequence having at least 89% homology to any one of SEQ ID Nos.: 1-11; and (b) culturing the cell from step (a) such that a protein encoded by the artificial vector is expressed, thereby synthesizing acyl CoA.

According to another aspect, there is provided a method for synthesizing acyl CoA comprising contacting CoA with an acyl group in the presence of a protein comprising an amino acid sequence with at least 93% homology to any one of SEQ ID Nos.: 12-22, thereby synthesizing acyl CoA.

According to another aspect, there is provided a method for obtaining an extract from a transgenic cell or a transfected cell comprising the steps: (a) culturing a transgenic cell or a transfected cell in a medium, wherein the transgenic cell or the transfected cell comprises a nucleic acid sequence having at least 89% homology to any one of SEQ ID Nos.: 1-11; and (b) extracting the transgenic cell or the transfected cell, thereby obtaining an extract from the transgenic cell or the transfected cell.

According to another aspect, there is provided an extract of a transgenic cell or a transfected cell obtained according to the herein disclosed method.

According to another aspect, there is provided a medium or a portion thereof separated from a cultured transgenic cell or a cultured transfected cell, obtained according to the herein disclosed method.

According to another aspect, there is provided a composition comprising: (a) the extract disclosed herein; (b) the herein disclosed medium or a portion thereof; or (c) a combination of (a) and (b), and an acceptable carrier.

In some embodiments, the nucleic acid sequence has at least 89% homology to any one of SEQ ID Nos.: 1-11 is 1,200 to 2,500 nucleotides long.

In some embodiments, the nucleic acid sequence encodes a protein characterized by acyl activating enzymatic activity.

In some embodiments, the isolated protein comprises an amino acid sequence with at least 93% homology to any one of SEQ ID Nos.: 12-22.

In some embodiments, the isolated protein consists of an amino acid sequence of any one of SEQ ID Nos.: 12-22.

In some embodiments, the isolated protein is characterized by acyl activating enzymatic activity.

In some embodiments, the acyl is selected from the group consisting of: C1-C8 alkyl chain, and alpha-unsaturated phenylalkyl carboxylic acid.

In some embodiments, the C1-C8 alkyl chain is hexanoic acid.

In some embodiments, the alpha-unsaturated phenylalkyl carboxylic acid comprises cinnamic acid or a derivative thereof.

In some embodiments, the cinnamic acid derivative is a hydroxylated derivative of cinnamic acid.

In some embodiments, the hydroxylated derivative of cinnamic acid is coumaric acid.

In some embodiments, the transgenic cell is any one of: a unicellular organism, a cell of a multicellular organism, and a cell in a culture.

In some embodiments, the unicellular organism comprises a fungus or a bacterium.

In some embodiments, the fungus is a yeast cell.

In some embodiments, the extract comprises the isolated DNA molecule, the isolated protein, or both.

In some embodiments, the transgenic plant is a Cannabis sativa plant.

In some embodiments, the protein is characterized by having an acyl activating enzymatic activity.

In some embodiments, the culturing comprises supplementing the cell with an effective amount of an acyl group.

In some embodiments, the acyl group is conjugated to the CoA so as to obtain the acyl CoA in the presence of the protein.

In some embodiments, the alpha-unsaturated phenylalkyl carboxylic acid comprises is cinnamic acid or a derivative thereof.

In some embodiments, the artificial vector is an expression vector.

In some embodiments, the cell is a prokaryote cell or a eukaryote cell.

In some embodiments, the cell is a transgenic cell, or a cell transfected with the isolated DNA molecule of the invention, or the artificial vector disclosed herein.

In some embodiments, the acyl CoA is selected form the group consisting of: acetyl CoA, butyryl CoA, hexanoyl CoA, octanoyl CoA, cinnamoyl CoA, coumaroyl CoA, and any combination thereof.

In some embodiments, the method further comprises a step preceding step (a), comprising introducing or transfecting the cell with the artificial vector.

In some embodiments, contacting is in a cell-free system.

In some embodiments, the method further comprises a step preceding step (b), comprising separating the cultured transgenic cell or the cultured transfected cell from the medium.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include graphs showing the identification of CBGA and heliCBGA in a H. umbraculigerum ethanolic extract. Extracted ion current (XIC) chromatograms and MS/MS spectral matching of (1A) CBGA (359.222 Da) and (1B) heliCBGA (393.206 Da) standards versus a H. umbraculigerum sample.

FIGS. 2A-2F include chemical structures illustrations and graphs showing isotope labeling of CBGA and heliCBGA via feeding of H. umbraculigerum leaves with hexanoic-D₁₁ acid, or phenylalanine-D₅ and phenylalanine-¹³C₉, respectively. H. umbraculigerum leaves were fed with either double distilled water (DDW, control), (2A) unlabeled/labeled hexanoic acid, or (2D) unlabeled/labeled phenylalanine for three days, then cannabinoids were extracted and analyzed via UPLC-qTOF. (2B and 2E) Extracted ion current (XIC) chromatograms and (2C and 2F) MS/MS spectra of CBGA and heliCBGA and their corresponding labeled peaks. The MS/MS spectra of the non-labeled versus the labeled forms show similar fragmentation patterns with mass shifts corresponding with the labeling. Since labeled metabolites possess nearly identical physicochemical properties as their native non-labeled analogues, the newly-derived iso-topologies were detected as co-eluting chromatographic peaks (unlabeled and labeled forms), except that their m/z values were different.

FIGS. 3A-3E include chemical structure illustrations and graphs showing the identification of CBGA-type alkyl homologues in a H. umbraculigerum ethanolic extract (3A) Extracted ion current (XIC) chromatograms of m/z 331.191, 345.207, 359.222, 373.238, and 387.254. The marked peaks in each chromatogram correspond with the detected C1-C7 compounds. As shown, the alkyl homologues elute from the reversed phase column in order of chain length as a result of increasing lipophilicities. MS/MS spectra in negative polarity of (3B) unlabeled and (3C) isotopically labeled compounds. Not all labeled compounds were detected probably due to low abundance. (3D) Suggested fragmentation structure of CBGA according to MS/MS spectra and labeling. Fragments colored in blue correspond to the m/z of the specific fragment in the compound labeled with hexanoic-D₁₁ acid. For all alkyl homologues, an appropriate m/z shift in the MS/MS spectra of all the product ions that include the alkyl chain was observed. (3E) Summary of the structures and the isotopically labeled precursors of the identified compounds.

FIGS. 4A-4B include chromatograms and a summary of the identified prenylated alkyl homologue phloroglucinoids in a H. umbraculigerum ethanolic extract. (4A) Extracted ion current (XIC) chromatograms of m/z 303.160, 317.175, 345.207, 359.222, 373.238, and 387.254. The marked peaks in each chromatogram correspond with the detected Ph1-Ph13 compounds. As shown, the alkyl homologues elute from the reversed phase column in order of chain length as a result of increasing lipophilicities. (4B) Summary of the structures and the isotopically labeled precursors of the identified compounds.

FIG. 5 includes a phylogenetic tree of the cloned AAEs from H. umbraculigerum and AAEs from Arabidopsis thaliana (Shokey et al., 2003) and from Cannabis (Stout et al., 2012) plants. Sequences were aligned using MUSCLE and a Maximum Likelihood tree using the JTT distance matrix-based method was constructed using MEGA11 software. Bootstrap values are indicated at the nodes of each branch (100 replicates). CsAAE1 is highlighted and is the most active enzyme in Cannabis and is similar to HuAAE4 (SEQ ID NO: 4). HuAAE6 (SEQ ID NO: 6), a highly active enzyme, disclosed herein, is similar to long-chain acyl-CoA synthetases (LACS), known to act on larger substrates (e.g., 16-30 carbon-long fatty acids).

FIG. 6 includes vertical bar graphs showing recombinant enzyme assays of purified H. umbraculigerum acyl activating enzyme (HuAAE) proteins produced in Escherichia coli cells. Peak area is shown. Various alkyl (short-and medium-chain fatty acids) and aromatic (cinnamic and coumaric acids) substrates were used in the enzyme assays.

### DETAILED DESCRIPTION

The present invention, in some embodiments, is directed to polynucleotide sequences derived from Helichrysum umbraculigerum and encoding a protein or a plurality thereof belonging to the acyl activating enzyme (AAE) family, including methods of using same.

According to some embodiments, there is provided a polynucleotide comprising a nucleic acid sequence comprising any one of SEQ ID Nos.: 1-11, or any combination thereof (“polynucleotide of the invention”).

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single- stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the polynucleotide comprises or consists of the nucleic acid sequence:

(SEQ ID NO: 1)
ATGACGTCGTCAAAGAAGTTTACAGTTGAAGTTGAACCGGCGATT
CCGGCCAAGGATGGAAAACCGTCGGCTGGACCGGTTTACCGTAGT
ATCTTTGCTAAAGACGGTTTTCCAGCTCATATTGACGGTTTAGAT
TCATGTTGGGATATTTTCCGCCTATCTGTGGAGAAATACCCCAAT
AATCGAATGCTTGGCACCCGTGAATTTGTGAATGGAAAGCATGGA
CCATATGTATGGTCGACTTACAAACAAGTATACGACAAGGTGATA
AAGGTTGGAAATGCTATCCGTGCGTGTGGTGTCGAGCCAGGTGGT
CGGTGTGGGATCTATGGTGCCAATTGTGCAGAATGGATTATGAGC
ATGGAGGCATGTAATGCTCATGGGCTTTACTGTGTACCTTTATAC
GATACCTTAGGTGCTGGTGCAATTGAATTCATTCTTTGCCATGCC
GAGGTTACAATTGCTTTTGTAGAAGAGAAAAAGATCCCTGAGTTG
TTGAAAACATTTCCGAAAGCTGGAGAATTTCTGAAAACAATTGTG
AGCTTTGGAAAAGTTACTCCTGAACAAAGAGAACAAGCTGAAAAC
TTTGGTTTAAAAATACATTCATGGGATGAATTCTTGACATTGGGT
GATGATAAAAACTTTGACCTGCCACTGAAGGAAAAAACTGATATC
TGTACAATAATGTACACTAGTGGAACAACTGGTGATCCTAAGGGT
GTTCTGATTTCAAATAACAGCATGGCAACACTTATAGCTGGCGTC
AATCGTCTACTAGATAGTGCAAAAGAATCTTTGAATCAACATGAT
GTCTATCTCTCGTTTTTACCTCTGGCACATATATTTGACCGTGTG
ATTGAAGAATGTTTTATCAATCATGGAGCATCTATAGGATTCTGG
CGTGGGGATGTTAAATTGCTGATTGAAGACATAGGGGAGCTGAAA
CCTACTATTTTCTGCGCTGTTCCTCGAGTGTTGGATAGGATTTAT
TCAGGTTTGCAACAGAAAATTTCTGCGGGGGGTTTTATCAAACGT
AACTTATTTAATCTAGCCTATTCATACAAATTACGTAATATGAAG
GGAGGGAAAACACATTCAGAGGCATCTCCATTGAGTGACAAAATC
GTCTTCAGTAAGGTTAAGCAGGGCCTAGGAGGAAATGTACGAATT
ATTCTATCTGGAGCTGCTCCACTAGCTCCACATGTAGAAGCTTAC
CTGAAAGTAGTGGCATGTAGTCACGTCCTGCAAGGATATGGCCTG
ACAGAAACTTGTGCTGGATCATTTGTCTCACTGCCAAACGAAATG
GAGATGCTGGGTACAGTGGGCCCACCTGTACCAGTTTTGGATGCC
CGACTGGAGTCTGTTCCGGAGATGAACTATGATGCTTGTTCAAGC
AAACCACAAGGAGAAATATGTATTAGAGGGGATGTTCTGTTTTCA
GGATACTACAAGCGTGAGGACCTTACAAAAGAAGTCTTTGTTGAT
GGGTGGTTCCATACAGGTGATATCGGTGAGTGGCAACCAGATGGA
AGCATGAAAATTATTGACCGAAAGAAAAACATTTTTAAGCTCTCA
CAAGGAGAGTACGTCGCAGTTGAAAATCTGGAGAATGTTTATGGA
AATGTTTCTGACATTGACACGATATGGATATATGGGAACAGCTTC
GAGTTTTGTCTTGTTGCTGTGGTCAACCCAAATGAGCCAGCAATC
AAACGTTATGCTGAAGCAAATAATATTTCTGGGGATTTTGATTCA
TTATGTGAAAATCCCAAAATTAAAGAATACATACTCGGAGAGCTC
GCTAGAATTGGAAAAGAGAAAAAGTTAAAAGGTTTTGAATTCGTC
AAAGCTGTTCACCTTGACCCTGTCCCTTTCGACATGGAACGTGAC
CTTCTGACCCCAACATTCAAGAAGAAAAGGCCCCAGATGCTTAAG
TACTACCAGGATGTAATTGATAACATGTACAAGACTATTAACAAG
AAGTGA.

In some embodiments, the polynucleotide comprises a nucleic acid sequence with at least 89%, at least 92%, at least 95%, or at least 97% homology or identity to SEQ ID NO: 1, or any value and range therebetween. Each possibility represents a separate embodiment of the invention. In some embodiments, the polynucleotide comprises a nucleic acid sequence with 89% to 100%, 90% to 100%, 95% to 100%, or 97% to 100% homology or identity to SEQ ID NO: 1. Each possibility represents a separate embodiment of the invention.

In some embodiments, the polynucleotide comprises or consists of the nucleic acid sequence:

(SEQ ID NO: 2)
ATGGATGCATTGAGGAAGCCTAATTCTGCGAATTCAAGCCCTTTA
ACTCCTATCGGATTCCTTGAAAGGGCAGCCGTCGTATTTGCCAAC
TCTCCTTCGATCGTATACAACAATCTCATCTACACTTGGAGCGAT
ACTTTTCATCGTTGTCTACGATTAGCTTCATCCATCTCTCGTCTC
GCTATACGAAAAGGCGACGTTGTTTCAGTACTCGCACCAAACATC
CCTGCCATTTATGAGCTTCATTTTGGCATCACTATGACTGGGGCC
ATAATCAACACCATCAATACCCGTTTGGATGCGCGTACTATCTCA
ATACTCCTTTGTCACAGTGAATCCAAGCTCGTCTTTGTTGATTAC
CAGTTGACTCGTCTTATACGAGAAGCGGTTTCTTTGATGCCAGAT
GCTTGTGTTCCCCCACAACTCGTCCTCATCGTAGATGACGGACAT
AATCTATCTTTACTTTCTGATCAATTTATCAATACTTATGAAGCT
ATGGTTGAAACAGGGGATCCTGGGTTCAATTGGGTTCGTCCAGAT
AGCGATTGGGACCCTCTAACGTTGAATTACACTTCTGGGACGACT
TCTTCCCCCAAAGGTGTTGTTAACAGCCACCGTGGATCGTTCATA
GTAGCGTTTGATTCTTTACTGGAGTGGCACGTACCGAAACAGCCG
ATCATGCTGTGGACTCTACCAATGTTCCACGCAAATGGGTGGAGC
TTCGTTTGGGGTATGGCAGCTGTTGGTGGCACCAATGTTTGCCTT
CGTAAATTCGATGCTACTATTATTTATGACACCATTCGTAACCAC
CATGTGACGCACATGTGTGGCGCCCCTGTTGTACTCAACATGTTA
TCAGAAGGTAAGCCACTTGAACACACGGTTCACATAATGACAGCA
GGAGCACCACCTCCAGCGGCCGTTTTGTTGCGAACCGAGTCGCTA
GGGTTTGAGGTGACTCATGGGTTCGGGATGACAGAAACAGGCGGG
TTAGTTGTGTCATGCTCATGGAAGAAAGAATGGAATCGTCTGCCC
GTGACTGAGAAAGCGAGATTGAAAGCGAGACAAGGAGTTAGAACA
CTTGGGATGACGGAAGTGGATATTGTGGATCCCGAGTCAGGAGTA
AGTGTGACTCGAGACGGGTTAACTCAGGGGGAATTAGTGTTGCGA
GGTGGGTCTATTATGTTGGGTTACTTAAAAGATCCGGAAACAACA
AATAAATCCGTTAAAAACGGGTGGTTTTATACCGGCGACGTGGCG
GTGATGCATCCAGATGGATATCTGGAAATAAAAGATAGATCAAAA
GATGTAATAATAAGTGGTGGTGAGAATATAAGTAGTGTGGAGGTT
GAGTCAATCTTGTATCAGCATCCTGCGATTAACGAGGCCGCGGTG
GTGGGACGGCCTGATGAGTTTTGGGGCGAGTCGCCGTGTGCTTTC
GTGAGTTTGAAAGATGATAACGGGAAGGTGGCTGTGCCAACAGCG
GATGAGATAATGAAGTTTTGTAAAGGAAAGTTGCCGGGTTACATG
GTACCCAAATCGGTTGTGTTTAAGAAGGATCTTCCGAAGACATCT
ACCGGTAAGATTCAGAAATATGTGCTTAGAAAACTTGCTAAAGAT
TTGGGTTTTGCTGTAAAAAGTCGAATTTAG.

In some embodiments, the polynucleotide comprises a nucleic acid sequence with at least 79%, at least 83%, at least 87%, at least 89%, at least 92%, at least 95%, at least 97%, or at least 99% homology or identity to SEQ ID NO: 2, or any value and range therebetween. Each possibility represents a separate embodiment of the invention. In some embodiments, the polynucleotide comprises a nucleic acid sequence with 79% to 100%, 80% to 100%, 82% to 100%, or 90% to 100% homology or identity to SEQ ID NO: 2. Each possibility represents a separate embodiment of the invention.

In some embodiments, the polynucleotide comprises or consists of the nucleic acid sequence:

(SEQ ID NO: 3)
ATGACCGAAGAGGAAAAAAATAAAGCAGAGTCCATGGGGATAAAA
ACGTATGCATGGAGCGACTTCCTTCATCTGGGGAGTAAAAATCCT
TCAGAACTGCAAACGCCTAAAGCAACTGATATATGTACAATCATG
TACACTAGTGGCACTAGTGGAGACCCAAAAGGTGTTATATTGACA
CATGAAAATGCTACAACAAACATACGAGGGGTTGATCTTTTCATG
GAACAATTCGAGGACAAGATGACCGTGGATGACGTTTATATATCT
TTCTTGCCTCTTGCTCACATTCTTGATCGTATGATTGAAGAATAC
TTTTTCCGTAGTGGTGCCTCTGTCGGCTTCTATCATGGGGATATC
AATGCGTTGAAGGAGGATTTGGCAGAGCTAAAGCCTACTTTTTTG
GCTGGAGTACCTCGAGTTTTGGAAAAGATTCACGAAGGTGTGCTT
AAAGGACTAGAAGAAGTTAATCCAAGGAGAAGGAAAATATTTAGC
ATTTTATACAATCACAAACTAAAATACATGAAAGCAGGTTACAAG
CATAAATATGCATCACCACTTGCAGATCTGCTTGCTTTTAGAAAG
GTTAAGAACAGGCTTGGTGGGCGAATTCGTCTTATGGTATCTGGA
GGAGCTCCGTTAAGCACTGAGATTGAAGAGTTCATGAGGGTTACT
TCATGTGCTTTTGTGGCGCAAGGATATGGTTTGACGGAAACATGT
GGTTTGGCTACTTTAGGATTTCCAGATGAGATGTGCATGATTGGA
ACAGTTGGTTCGCCCTTCGTGTATACAGAATTACGCCTCGAAGAA
GTTTCAGATATGGGCTATGACCCGTTGGCCAATCCACCACGTGGT
GAAATATGTGTTAAGGGAAAAACGCCTTTCGCAGGTTACTACAAG
AATCCAGAACTCACTAATGAGGTCATGAAAGATGGGTGGTTTCAT
ACAGGTGACATAGGAGAGATGCAACCAAACGGGGTATTGAAAATC
ATCGACAGAAAGAAACATCTGATAAAACTATCTCAAGGGGAGTAT
ATCGCGCTTGAATATCTAGAGAAAGTTTACTGCATCACTCCCATT
CTTGAAGACATCTGGGTATATGGGGATAGCTTCAAGTCATCATTG
GTCGCGGTAGCTGTACCAAACAAAGAAAACGCAGAAAAGTGGGCC
GATCAAAAGGGCCTTAAAGTTTCTTACTCTGAGCTCTGCACACTA
ACACAGTTCAGAGATTATATCCAATCTGAACTGAAATCTACCGCG
GAGAGAAACAAGCTAAGAGGTTTTGAGCATATAAAGGCTATAATT
GTGGAGCCACGGACGTTTGAAGGAGACCAGGAATTGTTGACTGCA
ACAATGAAGAAACGTAGAAATAAACTGCTTAACCGTTACAAGGAG
GGGATCGACAACCTTTACAAGAACTTGGCTGCAAACAAACGCTG
A.

In some embodiments, the polynucleotide comprises a nucleic acid sequence with at least 86%, at least 88%, at least 90%, at least 95%, at least 97%, or at least 99% homology or identity to SEQ ID NO: 3, or any value and range therebetween. Each possibility represents a separate embodiment of the invention. In some embodiments, the polynucleotide comprises a nucleic acid sequence with 86% to 100%, 88% to 100%, 90% to 100%, or 92% to 100% homology or identity to SEQ ID NO: 3. Each possibility represents a separate embodiment of the invention.

In some embodiments, the polynucleotide comprises or consists of the nucleic acid sequence:

(SEQ ID NO: 4)
ATGGTGTACAAGTCTTTGAATTCAATATCCATATCAGATATAGTA
AATCTTGGTATATCACCTGAAACTGCAACTCAACTTCATCAGAAA
CTAACTGAAATCATTCAGATTTATGGTTTTGATGCTCCTCAAACA
TGGACCCAGATATCCACCCGGATTCTTCATCCGGACCTTCCCTTT
TGTTTTCATCAGATGATGTATTATGGATGCTATGTTGATTTTGGA
CCGGATCCTCCTGCTTGGTCACCCGACCCGAAGGATGCAAAGTTA
ACAAACATAGGTAGTTTATTAGAGAGACGCGGAAAGGAGTTCTTG
GGGCCTAGTTATAAAGATCCCATTTCAAGCTACTCTGCTCTTCAG
GAATTTTCAGCCTTAAATCTAGAGGTGTTTTGGAAAACAATATTG
GATGAAATGAATATAACATTTTCTGTGCCTCCAAAACGCATATTA
GTTGATGACCTGTCTAAAGAAAGCCAGTTATTGCATCCAGGTGGT
CGATGGCTTCCCGGAGCTTATGTAAATCCAGCTAGAAATTGTTTG
AGTTTAAGTAGCAAGAGAAGGTTAAGTGATATAGCAGTTATATGG
CGTGATGAAGGAAATGATGATATGCCGGTCAACAAAATGACGTTT
CAGCAGTTGCGCTCAGAGGTTTGGTTAGTTGCATATGCACTTGAT
ACATTGGGAGTGGAAAAAGGATCTGCAATTGCAATCGATATGCCT
ATGGATGTCAAATCTGTGGTGATTTATCTAGCCATTGTTTTAGCA
GGCTATGTGGTTGTATCTATTGCAGATAGTTTTGCTGCTGGTGAA
ATTTCGACCAGACTTGTATTATCAAAAGCAAAAGCAATTTTTACT
CAGGATTTGATCATTCGTGGTGACAGAAGCCATCCCTTGTACAGC
CGAGTTGTTGATGCTCAATCACCTCTAGCAATTGTCATTCCTACG
AGAGGCTCAAGTTTTAGTATAAAATTACGTGACGGTGATATTTCT
TGGCATGATTTTCTGGAACGAGCTAACACTTACAGGAATGTTGAG
TTTGTTGCTGTTGAACGACCCGTTGAAGCTTTCTCAAATATCCTT
TTCTCATCAGGAACTACAGGGGAACCGAAGGCAATTCCATGGACC
CTTGCAACACCTTTCAAGGCTGGTGCAGACGCTTGGTGCCACATG
GATGTCCACAAAGGTGATGTTGTTGCATGGCCTACTAATCTTGGA
TGGATGATGGGTCCTTGGCTAATATATGCTTCATTGTTAAATGGG
GGCTCACTTGCATTATACAACGGATCTCCCCTGACTTCTGGATTT
GCCAAGTTTGTTCAGGATGCAAAAGTAACATTGTTGGGAGTGATA
CCAAGTATTGTGAGGGCATGGAGAACAAACAATAGTACAGCCGGC
TTTGACTGGTCAACCATCCGGTGCTTTGGATCGACCGGTGAGGCC
TCTAATACTGATGAATGTCTTTGGCTGATGGGAAGAGCTCATTAC
AAACCGGTCATCGAGTATTGCGGTGGCACAGAGATTGGTGGTGGT
TTTATTACAGGATCTTTACTGCAGCCTCAGTGTTTGTCTGCTTTC
AGCACACCAAGTTTGGGTTGTAAACTGTTAATTCTTGGCGAAGAT
GGAATCCCTATACCACAAAACGCTCCTGGAATTGGTGAATTGGCT
CTGAATCCCCTCATGTTTGGGGCATCGAGCACACTACTAAATGCA
AACCACTATGATGTCTACTTTAAAGGCATGCCCTCTTGGAATGGT
AAGGTTCTAAGAAGGCATGGAGATGTATTTGAGCGCACGTCTAAA
GGATACTATCGTGCCCATGGTCGTGCAGATGATACTATGAATCTT
GGGGGTATTAAGGTAAGTTCGGTTGAGATTGAACGTGTATGCAAC
TCGATTGATGACAGAATTCTCGAGACAGCGGCTATAGGGGTTACA
CCTTCTGGTGGCGGGCCAGAGAGGTTGGTAATTGTTGTTGCTTTT
AAAGATGGCAGTGGTTCGAAACCCGACTTAATCAAGTTGAAGGTC
ACACTGAATTCAGCTTTACAAAAGAATCTGAACCCTTTGTTTAAG
GTTTCTGATGTGGTGCCCTTTCCATCACTTCCTAGGACAGCAACA
AACAAGGTAATGAGAAGGGTTTTGCGACAGCAGTTGACTCAAATT
GGTCAAAATAGCAAGCTATAA.

In some embodiments, the polynucleotide comprises a nucleic acid sequence with at least 88%, at least 90%, at least 92%, at least 95%, at least 97%, or at least 99% homology or identity to SEQ ID NO: 4, or any value and range therebetween. Each possibility represents a separate embodiment of the invention. In some embodiments, the polynucleotide comprises a nucleic acid sequence with 88% to 100%, 90% to 100%, 92 to 100%, or 95% to 100% homology or identity to SEQ ID NO: 4. Each possibility represents a separate embodiment of the invention.

In some embodiments, the polynucleotide comprises or consists of the nucleic acid sequence:

(SEQ ID NO: 5)
ATGGGTGATTCAGAGGGAAGCAGCATTAGTACTCCTACAACTGAA
CAAGTTGGTTTCTTGTCAAATATCATGGAAGACAAATCTTATAGT
GCTGCAGTTGCAATTATGGTTGCCATTGCTGTACCGTTGGTTCTT
TCTTCAGTGTTTGCAGCGAAGAAGAAAGTGAAACAACGAGGCGTT
CCCGTTCAAGTTGGTGGTGAGCCAGGTTTTGCCATGCGTAACTCT
AGATCAAACAAATTAGTTGATGTCCCATGGGAAGGAGCTAGAACA
ATGGCTGCTCTTTTTGAGCAGTCTTGTAAGAAGCATTCACAGCTT
CGGTTTCTTGGTACAAGGAAGTTGATTGAAAGAAGCTTTGTGAGT
GGTAGTGATGGGAGAAAATTCGAGAAGTTACATCTTGGGGAGTAT
CAGTGGGAGACATATGGGCAGATATTTGAACGTGTTTGCAACTTT
GCATCTGGACTTATTCAGCTTGGTCATGACCCTGATACTCGTATT
GCCATCTTTTCTGACACACGAGCTGAATGGTTAATTGCATTTGAG
GGATGCTTCAGGCAGAACATCACTGTGGTTACCATATATGCATCA
TTAGGTGATGATGCCCTCATTCACTCTCTTAACGAGACTAAAGTA
TCGACCTTGATTTGTGATTCCAAACTATTGAAAAAAGTGGCTGCA
GTTAGTTCAAGCCTGAAAACTGTAGAAAACTTCATCTACTTTGAA
AGTGACAACACTGAAGCTTTAAATGAAATCGGTGATTGGAAAATA
TCTTCTTTTTCTGAAGTCGAGAGCTTGGGACAGAAGAGTCCAGTA
AGTGCTAGACTGCCTATCAAGAAAGACGTTGCAGTGATCATGTAT
ACAAGTGGCAGCACAGGTTTACCAAAGGGGGTGATGATGACTCAT
GGGAATGTAGTAGCAACTGCAGCTGCGGTTATGACTGTAATCCCA
AATATTGGGACCAATGATGTTTATCTGGCATACTTACCATTGGCT
CATATTTTCGAGTTGGCTGCTGAGACTGTGATGGTAACTGCAGGT
ATTCCAATTGGTTATGGTTCAGCACTCACTTTAACAGACACATCA
AATAAAATCAAGAAAGGAACCTTGGGAGATGCATCCATCTTGAAG
CCAACGTTAATGGCAGCTGTTCCAGCTATTTTAGATCGTGTCCGA
GATGGAGTATTAAAGAAGGTTGAGGAAAAGGGAGGTTTGACAACA
AAAATATTCAATATAGCCTACAAAAGGCGTTTGCTAGCAGTAGAT
GGAAGTTGGCTGGGTGCATGGGGGTTAGAGAAGCTATTGTGGGAT
GCCATTGTTTTTAAGAAGATTCGTTCTGTACTTGGAGGAGATATC
CGTTTCATGCTCTGTGGTGGTGCTCCTTTAGCTGCAGATACTCAG
CGATTTATAAATGTCTGCGTTGGGGCTCCAATTGGACAAGGATAT
GGGCTGACCGAAACATGCGCTGGAGCTGCTTTCTCTGAGGCAGAT
GATAATTCTGTTGGGCGTGTTGGTCCACCACTTCCTTGTGTCTAT
ATTAAACTTGTTTCATGGGATGAAGGTGGGTATTTAACATCAGAC
AAACCAATGCCGCGAGGCGAAGTTGTAGTTGGTGGGTACAGTGTA
ACCGCTGGTTACTTTAATAATGAGGAAAAGACCAATGAGGTTTAC
AAGGTTGATGAAAGTGGGATGCGTTGGTTCTACACTGGGGACATT
GGAAGGTTTCATCCTGATGGATGCCTTGAAATCATTGACAGGAAG
AAGGATATTGTAAAACTTCAACATGGAGAGTACATCTCCTTGGGG
AAGGTTGAGGCAGCACTTGCGTCAAGCAAGTATGTAGAGAATGTA
ATGTTACATGCCGACCCCTTCCACACTTATTGTGTCGCCTTAGTT
GTCCCTGCGCGTCAGGTTATAGAACAGTGGGCTCAAGATGCGGGT
ATTAGTTACCAAGATTTTGCTGAGTTGTGTGATAAAAAGGAAACT
GTCTCTGAGGTTCAGCAATCCCTTACCAAGGTAGCAAAAGATGCA
AAACTAGACAAGTTTGAAACGCCTGCAAAGATAAAGCTGATGCCA
GATCCATGGACTCCTGAATCTGGATTAGTAACAGCGGCTCTTAAG
TTAAAAAGGGAACAACTGAAGTCCAAATTTAAGGATGATCTGGAT
AAGCTATATGGGTGA.

In some embodiments, the polynucleotide comprises a nucleic acid sequence with at least 88%, at least 90%, at least 92%, at least 95%, at least 97%, or at least 99% homology or identity to SEQ ID NO: 5, or any value and range therebetween. Each possibility represents a separate embodiment of the invention. In some embodiments, the polynucleotide comprises a nucleic acid sequence with 88% to 100%, 90% to 100%, 91 to 100%, or 95% to 100% homology or identity to SEQ ID NO: 5. Each possibility represents a separate embodiment of the invention.

In some embodiments, the polynucleotide comprises or consists of the nucleic acid sequence:

(SEQ ID NO: 6)
ATGTCGGTTTACACCGTTAAAGTCGAGGATTCACGGGCAGCTTCC
GGAGAAACCCCGTCAGCAGGGCCGGTTTACAGGTGCATTTATGCC
AAGGATGCTCTCATGGAACTGCCCCCCGGTTATGAATCTCCCTGG
GACTTCTTTAGTGAGTCTGTTAAAAGAAACCCAAAGAACCCAGCA
CTAGGTCGTCGTCAAGTCATCGATGGAAAGGCTGGTGGTTATTCA
TGGCTTTCATATCAAGAAGCCTACAATTCTGCTCTACGCATTGCT
TCTGCCATCAGAAGCCGATCTGTTAATCCTGGGGATCGGTGTGGT
ATATATGGACCTAACTGTCCTGAATGGATAATCTCAATGGAGGCT
TGTAACAGCAATGGCATAACCTATGTTCCCCTATATGATACACTT
GGTGCTAATGCGGTTGAATACATCATCAACCATGCAGAAATTTCT
TTAGTTTTTGTTCAAGAGAACAAGTTGTCTGCTATTTTATCATGT
CTTCCAAATTGCTCATCAAATCTTAAAACAATCGTCAGCTTTGGG
AAGTTCTCTGAATCACAAAAGAACGAAGCCATGGAACATGGCGTC
GATTGCTTCTCTTGGGAAGAGTTTTCTTCGATGGGGAATTTGGAA
GATGAACTTCCTGCAAAAAATAAGACTGACATTTGCACCATAATG
TATACAAGTGGAACAACGGGAGAGCCTAAGGGTGTCGTACTAAGT
AACAGAGCTTTCATGTCCGAAGTCTTGTCTATGCATGAACTACTC
ATAGAAACAGACAAACCGGGCACAGAAGAAGATACCTACTTCTCT
TTTCTTCCTTTGGCACATATATTTGATCAAATAATGGAGACGTAT
TTCATCTACAGTGGTGCTTCGATAGGGTTTTGGCAAGGAGATATC
AGATACTTGATTGAAGACCTTCTTGTGTTGCAGCCAACCATATTT
TGTGGTGTTCCAAGAGTTTATGACCGCATTTATACGGGCATAATG
GCTAAGATTTCAACTGGAGGTGCTATTCGGAAGGCATTATTTGAT
TTTGCATACAACTATAAATTAAGGAACCTTGAAAAGGGAATACAA
CAAGACAAATCAGCTCCTCTTTTGGACAAGCTGGTCTTCGATAAG
ATTAAACAAGGGTTTGGAGGAAGGGTTCGTCTTATGTTATCTGGA
GCCGCACCTTTGCCAAAACACGTGGAGGAATTTTTAAGAGTGACG
TGCTGTACCGTTCTCTCACAAGGATACGGACTTACTGAAAGTTGT
GGTGGATGCTTTACATCCATTGCGAATGTGTACTCTATGATCGGG
ACTGTTGGTGTACCCATGACAACTATTGAAGCAAGACTTGAGTCA
GTGCCAGAGATGGGATATGATGCACTCAGTAGTGTGCCATGTGGC
GAAATTTGCCTCAGGGGAAACACACTATTTTCTGGGTACCACAAA
CGAGACGATCTAACTGATGCTGTCCTTGTAGATGGCTGGTTCCAT
ACAGGTGACATTGGGGAATGGCAGGCAGATGGAGCAATGAAAATC
ATTGACAGGAAAAAGAATATATTCAAATTGTCTCAAGGAGAATAT
GTTGCAGTTGAAAGTATTGAAAGCACCTATTCACGGTGTCCTTTG
GTTACCTCGATTTGGGTGTACGGCAATAGTTTTGAATCTTTTCTA
GTTGCGGTTGTGGTTCCCGATAGAGTAGCAGTTGAAGAGTTTGCT
GCAAAGAACAATGAATCAGGAGATTATGCATCGTTGTGCAAGAAC
CCAAATGTCAGGAAATATGTTCTTGAAGAGCTGAATGCTGAAGCT
CAATGCAATAAACTTCGCGGGTTTGAGATGCTAAAAGCAGTTCAT
TTGGATCCAGTCCCATTTGACTTCGAGAGGGATTTAATAACACCA
ACCTTTAAACTAAAAAGACAGCAGCTTCTAAAATACTATAAGGAT
TGCGTTGAACAACTATATGCTGAAGCAAAGACATCCAAGAAATGA.

In some embodiments, the polynucleotide comprises a nucleic acid sequence with at least 89%, at least 90%, at least 92%, at least 95%, at least 97%, or at least 99% homology or identity to SEQ ID NO: 6, or any value and range therebetween. Each possibility represents a separate embodiment of the invention. In some embodiments, the polynucleotide comprises a nucleic acid sequence with 89% to 100%, 90% to 100%, 93 to 100%, or 95% to 100% homology or identity to SEQ ID NO: 6. Each possibility represents a separate embodiment of the invention.

In some embodiments, the polynucleotide comprises or consists of the nucleic acid sequence:

(SEQ ID NO: 7)
ATGGAAACTCATGGACCAAGGCTTCTAGGTGCAGCTTACAAAGAT
CCTATCACGAGTTATAAACAGTTCCAAAAGTTCTCTGTTCAACAT
CTAGAGGTGTATTGGTCTCTTGTGTTAGAAAAGCTTTCAATCCAA
TTTCAGGAACGTCCAAAATGTATAGTAGATACTTCTGACAAATCA
AAACACGGGGGCACATGGCTTCCCGGTTCAGTTTTGAACATTGCG
GAGTGTTGTATATTGTCAACTACTGAAACAGATGAAAAGGTTGCG
ATTGTGTGGCGGGATGAAAGATGTGATAATCTGGATGTAAACAAG
ATGACATTCAAAGAATTGCGACAACAAGTAATGTTGGTTGCAAAT
GCATTGAAGTTATTGTTTTCAAAAGGAGATCCTATTGCAATTGAT
ATGCCAATGACAGTTACTGCAGTAATTCTATATTTGGCGATTGTA
TATTCTGGATTTGTGGTTGTATCTATAGCTGACAGTTTTGCAGCT
AAAGAGATTGCAACACGATTACGTGTATCTAATGCAAAGGCTATC
TTTACTCAAGATTACATTGTTCGAGGTGGTCGAAGATTTCCTTTG
TACAGTCGAGTTATTGAAGCCACCCAATGTAGAGCCATCGTGGTT
CCTGCGATAGGGGAAAACGTAGAAGTTATTTTAAGAAAACAGGAC
ATTTCATGGGGCGATTTTCTTTCTGGTGCAAAACAGCTTCCTAGC
CCGGATTATTGCTCTCCAGTCTATCAATCCATAGACACGTTGACA
AACATACTCTTCTCTTCGGGAACAACAGGAGACCCAAAAGCTATA
CCATGGACGCAAATATCTCCAATGAGATGTGCTGCTGACGGATGG
GCTCATATGGATATTCAGGCTGGAGATGTTTATTGTTGGCCCACA
AATCTGGGATGGGTCATGGGACCCATTGTACTTTACTCGAGTTTT
CTTACCGGTGCAACATTGGCTCTTTATAATGGCTCCCCTCTTGGT
CATGGTTTTGGAAAATTTGTTCAGGATGCAGGAGTGACAATTTTG
GGCACGGTTCCAAGCATAGTCAAGTCTTGGAAGAGTACAAGATGT
ATGGAAGGACTGGACTGGACAAAGATAAAGGCATTTGGGTCGACT
GGTGAAGCTTCTAATGTCGACGATGACCTTTGGCTTTCCTCAAAG
GCCTACTACAAACCTGTTCTTGAATGCTGTGGAGGTACCGAGCTT
GCATCTTCTTATGTTCAAGGGAATCTTCTACAGCCACAAGCCTTT
GGAGCATTAAGCTCTGCTTCAATGGGAACCGGATTTGTCATATTT
GACGATCATGGAGTTCCTTACCCGGACGATGAACCCTGTGTTGGT
GAAGTGGGTTTGTTTCCAGTATATATGGGAGCATCTGATAGACTA
CTGAATGCAGATCATGAAAAAATTTACTTCAAGGGAATGCCGAGT
TACAAAGGAATGCAACTAAGGAGACATGGAGATATCATCAAGAGA
ACAATTGGAGGATATTTGGTTGTACAAGGCAGGGCTGATGATACC
ATGAACCTTGGTGGCATAAAGACGAGCTCAATAGAAATTGAGCGT
GTTTGTGAACAAGCTGATGGAAGCATCATGGAAACTGCTGCAGTC
AGTGTTGCACCTGCAACCGGTGGTCCAGAACTATTAGCCATATTT
GTGGTACTAAAGAACGGTTGCAACACTCAACCACAGGACCTAAAG
ATGATATTTTCAAAGGCCATTCAAAAAAACCTCAACCCATTGTTC
AAGGTGAGCTTTGTAAAGGTTGTTCCAGAGTTCCCTCGAACCGCT
TCTAACAAGTTATTGAGAAGAGTTTTAAGGAATCAAGTGAAGGAA
GAGCTTCAAACTCGAAGTAAAATATAA.

In some embodiments, the polynucleotide comprises a nucleic acid sequence with at least 85%, at least 87%, at least 90%, at least 95%, at least 97%, or at least 99% homology or identity to SEQ ID NO: 7, or any value and range therebetween. Each possibility represents a separate embodiment of the invention. In some embodiments, the polynucleotide comprises a nucleic acid sequence with 85% to 100%, 88% to 100%, 85% to 100%, or 92% to 100% homology or identity to SEQ ID NO: 7. Each possibility represents a separate embodiment of the invention.

In some embodiments, the polynucleotide comprises or consists of the nucleic acid sequence:

(SEQ ID NO: 8)
ATGGAGATCACTAAAAGCATCCAAGAATTAGGATTACAAGATCTACTAA
ACACTGGATTAACACCTAATGATGCAAAATCACTGCAAATCGAGATTAA
ACACATCATTAATAGTCAAACTACTAATTCAAACCCAGTTGAGTTATGG
CGTCAAATCACTTCTGCAAAGCTGCTTAAACCCTCTTATCCTCATTCGT
TGCACCAGCTCATCTACTACGCGGTGTACTGTAACTATGATGCATCCAT
CTATGGTCCTCCCCTGTATTGGTTTCCATCTGAAATTGATTCTAAAAGG
TCAAACTTGGGGAACATTATGGAAACTCATGGACCAAGGCTTCTAGGTG
CAGCTTACAAAGATCCTATCACGAGTTATAAACAGTTCCAAAAGTTCTC
TGTTCAACATCTAGAGGTGTATTGGTCTCTTGTGTTAGAAAAGCTTTCA
ATCCAATTTCAGGAACGTCCAAAATGTATAGTAGATACTTCTGACAAAT
CAAAACACGGGGGCACATGGCTTCCCGGTTCAGTTTTGAACATTGCGGA
GTGTTGTATATTGTCAACTAGTGAAACAGATGATAAGGTTGCGATTGTA
TGGCGGGATGAAAGATGTGATAATCTGGATGTAAACAAGATGACATTCA
AAGAATTGCGACAACAAGTAATGTTGGTTGCAAATGCATTGAAGTTATT
GTTTTCAAAAGGAGATCCTATTGCAATTGATATGCCAATGACAGTTACT
GCAGTAATTCTATATTTGGCGATTGTATATTCTGGATTTGTGGTTGTAT
CTATAGCTGACAGTTTTGCAGCTAAAGAGATTGCAACACGATTACGTGT
ATCTAATGCAAAGGCTATCTTTACTCAAGATTACATTGTTCGAGGTGGT
CGAAGATTTCCTTTGTACAGTCGAGTTATTGAAGCCACCCAATGTAGAG
CCATCGTGGTTCCTGCGATAGGGGAAAACGTAGAAGTTATTTTAAGAAA
ACAGGACATTTCATGGGGCGATTTTCTTTCTGGTGCAAAACAGCTTCCT
AGCCCGGATTATTGCTCTCCAGTCTATCAATCCATAGACACGTTGACAA
ACATACTCTTCTCTTCGGGAACAACAGGAGACCCAAAAGCTATACCATG
GACGCAAATATCTCCAATGAGATGTGCTGCTGACGGATGGGCTCATATG
GATATTCAGGCTGGAGATGTTTATTGTTGGCCCACAAATCTGGGATGGG
TCATGGGACCCATTGTACTTTACTCGAGTTTTCTTACCGGTGCAACATT
GGCTCTTTATAATGGCTCCCCTCTTGGTCATGGTTTTGGAAAATTTGTT
CAGGATGCAGGAGTGACAATTTTGGGCACGGTTCCAAGCATAGTCAAGT
CTTGGAAGAGTACAAGATGTATGGAAGGACTGGACTGGACAAAGATAAA
GGCATTTGGGTCGACTGGTGAAGCTTCTAATGTCGACGATGACCTTTGG
CTTTCCTCAAAGGCCTACTACAAACCTGTTCTTGAATGCTGTGGAGGTA
CCGAGCTTGCATCTTCTTATGTTCAAGGGAATCTTCTACAGCCACAAGC
CTTTGGAGCATTAAGCTCTGCTTCAATGGGAACCGGATTTGTCATATTT
GACGATCATGGAGTTCCTTACCCGGACGATGAACCCTGTGTTGGTGAAG
TGGGTTTGTTTCCAGTATATATGGGAGCATCTGATAGACTACTGAATGC
AGATCATGAAAAAATTTACTTCAAGGGAATGCCGAGTTACAAAGGAATG
CAACTAAGGAGACATGGAGATATCATCAAGAGAACAATTGGAGGATATT
TGGTTGTACAAGGCAGGGCTGATGATACCATGAACCTTGGTGGCATAAA
GACGAGCTCAATAGAAATTGAGCGTGTTTGTGAACAAGCTGATGGAAGC
ATCATGGAAACTGCTGCAGTCAGTGTTGCACCTGCAACCGGTGGTCCAG
AACTATTAGCCATATTTGTGGTACTAAAGAACGGTTGCAACACTCAACC
ACAGGACCTAAAGATGATATTTTCAAAGGCCATTCAAAAAAACCTCAAC
CCATTGTTCAAGGTTTTCTCCTAA.

In some embodiments, the polynucleotide comprises a nucleic acid sequence with at least 84%, at least 87%, at least 90%, at least 95%, at least 97%, or at least 99% homology or identity to SEQ ID NO: 8, or any value and range therebetween. Each possibility represents a separate embodiment of the invention. In some embodiments, the polynucleotide comprises a nucleic acid sequence with 84% to 100%, 88% to 100%, 90% to 100%, or 95% to 100% homology or identity to SEQ ID NO: 8. Each possibility represents a separate embodiment of the invention.

In some embodiments, the polynucleotide comprises or consists of the nucleic acid sequence:

(SEQ ID NO: 9)
ATGGTGTACAAGTCTTTGAATTCAATATCCATATCAGATATAGTAAATC
TTGGTATATCACCTGAAACTGCAACTCAACTTCATCAGAAACTAACTGA
AATCATTCAGATTTATGGTTTTGATGCTCCTCAAACATGGACCCAGATA
TCCACCCGGATTCTTCATCCGGACCTTCCCTTTTGTTTTCATCAGATGA
TGTATTATGGATGCTATGTTGATTTTGGACCGGATCCTCCTGCTTGGTC
ACCCGACCCGAAGGATGCAAAGTTAACAAACATAGGTAGTTTATTAGAG
AGACGCGGAAAGGAGTTCTTGGGGCCTAGTTATAAAGATCCCATTTCAA
GCTACTCTGCTCTTCAGGAATTTTCAGCCTTAAATCTAGAGGTGTTTTG
GAAAACAATATTGGATGAAATGAATATAACATTTTCTGTGCCTCCAAAA
CGCATATTAGTTGATGACCTGTCTAAAGAAAGCCAGTTATTGCATCCAG
GTGGTCGATGGCTTCCCGGAGCTTATGTAAATCCAGCTAGAAATTGTTT
GAGTTTAAGTAGCAAGAGAAGGTTAAGTGATATAGCAGTTATATGGCGT
GATGAAGGAAATGATGATATGCCGGTCAACAAAATGACGTTTCAGCAGT
TGCGCTCAGAGGTTTGGTTAGTTGCATATGCACTTGATACATTGGGAGT
GGAAAAAGGATCTGCAATTGCAATCGATATGCCTATGGATGTCAAATCT
GTGGTGATTTATCTAGCCATTGTTTTAGCAGGCTATGTGGTTGTATCTA
TTGCAGATAGTTTTGCTGCTGGTGAAATTTCGACCAGACTTGTATTATC
AAAAGCAAAAGCAATTTTTACTCAGGATTTGATCATTCGTGGTGACAGA
AGCCATCCCTTGTACAGCCGAGTTGTTGATGCTCAATCACCTCTAGCAA
TTGTCATTCCTACGAGAGGCTCAAGTTTTAGTATAAAATTACGTGACGG
TGATATTTCTTGGCATGATTTTCTGGAACGAGCTAACACTTACAGGAAT
GTTGAGTTTGTTGCTGTTGAACGACCCGTTGAAGCTTTCTCAAATATCC
TTTTCTCATCAGGAACTACAGGGGAACCGAAGGCAATTCCATGGACCCT
TGCAACACCTTTCAAGGCTGGTGCAGACGCTTGGTGCCACATGGATGTC
CACAAAGGTGATGTTGTTGCATGGCCTACTAATCTTGGATGGATGATGG
GTCCTTGGCTAATATATGCTTCATTGTTAAATGGGGGCTCACTTGCATT
ATACAACGGATCTCCCCTGACTTCTGGATTTGCCAAGTTTGTTCAGGAT
GCAAAAGTAACATTGTTGGGAGTGATACCAAGTATTGTGAGGGCATGGA
GAACAAACAATAGTACAGCCGGCTTTGACTGGTCAACCATCCGGTGCTT
TGGATCGACCGGTGAGGCCTCTAATACTGATGAATGTCTTTGGCTGATG
GGAAGAGCTCATTACAAACCGGTCATCGAGTATTGCGGTGGCACAGAGA
TTGGTGGTGGTTTTATTACAGGATCTTTACTGCAGCCTCAGTGTTTGTC
TGCTTTCAGCACACCAAGTTTGGGTTGTAAACTGTTAATTCTTGGCGAA
GATGGAATCCCTATACCACAAAACGCTCCTGGAATTGGTGAATTGGCTC
TGAATCCCCTCATGTTTGGGGCATCGAGCACACTACTAAATGCAAACCA
CTATGATGTCTACTTTAAAGGCATGCCCTCTTGGAATGGTAAGGTTCTA
AGAAGGCATGGAGATGTATTTGAGCGCACGTCTAAAGGATACTATCGTG
CCCATGGTCGTGCAGATGATACTATGAATCTTGGGGGTATTAAGGTAAG
TTCGGTTGAGATTGAACGTGTATGCAACTCGATTGATGACAGAATTCTC
GAGACAGCGGCTATAGGGGTTACACCTTCTGGTGGCGGGCCAGAGAGGT
TGGTAATTGTTGTTGCTTTTAAAGATGGCAGTGGTTCGAAACCCGACTT
AATCAAGTTGAAGGTCACACTGAATTCAGCTTTACAAAAGAATCTGAAC
CCTTTGTTTAAGGTTTCTGATGTGGTGCCCTTTCCATCACTTCCTAGGA
CAGCAACAAACAAGGTAATGAGAAGGGTTTTGCGACAGCAGTTGACTCA
AATTGGTCAAAATAGCAAGCTATAA.

In some embodiments, the polynucleotide comprises a nucleic acid sequence with at least 88%, at least 90%, at least 92%, at least 95%, at least 97%, or at least 99% homology or identity to SEQ ID NO: 9, or any value and range therebetween. Each possibility represents a separate embodiment of the invention. In some embodiments, the polynucleotide comprises a nucleic acid sequence with 88% to 100%, 90% to 100%, 93 to 100%, or 96% to 100% homology or identity to SEQ ID NO: 9. Each possibility represents a separate embodiment of the invention.

In some embodiments, the polynucleotide comprises or consists of the nucleic acid

sequence:

(SEQ ID NO: 10)
ATGACGTTTCAGCAGTTGCGCTCAGAGGTTTGGTTAGTTGCATATGCAC
TTGATACATTGGGAGTGGAAAAAGGATCTGCAATTGCAATCGATATGCC
TATGGATGTCAAATCTGTGGTGATTTATCTAGCCATTGTTTTAGCAGGC
TATGTGGTTGTATCTATTGCAGATAGTTTTGCTGCTGGTGAAATTTCGA
CCAGACTTGTATTATCAAAAGCAAAAGCAATTTTTACTCAGGATTTGAT
CATTCGTGGTGACAGAAGCCATCCCTTGTACAGCCGAGTTGTTGATGCT
CAATCACCTCTAGCAATTGTCATTCCTACGAGAGGCTCAAGTTTTAGTA
TAAAATTACGTGACGGTGATATTTCTTGGCATGATTTTCTGGAACGAGC
TAACACTTACAGGAATGTTGAGTTTGTTGCTGTTGAACGACCCGTTGAA
GCTTTCTCAAATATCCTTTTCTCATCAGGAACTACAGGGGAACCGAAGG
CAATTCCATGGACCCTTGCAACACCTTTCAAGGCTGGTGCAGACGCTTG
GTGCCACATGGATGTCCACAAAGGTGATGTTGTTGCATGGCCTACTAAT
CTTGGATGGATGATGGGTCCTTGGCTAATATATGCTTCATTGTTAAATG
GGGGCTCACTTGCATTATACAACGGATCTCCCCTGACTTCTGGATTTGC
CAAGTTTGTTCAGGATGCAAAAGTAACATTGTTGGGAGTGATACCAAGT
ATTGTGAGGGCATGGAGAACAAACAATAGTACAGCCGGCTTTGACTGGT
CAACCATCCGGTGCTTTGGATCGACCGGTGAGGCCTCTAATACTGATGA
ATGTCTTTGGCTGATGGGAAGAGCTCATTACAAACCGGTCATCGAGTAT
TGCGGTGGCACAGAGATTGGTGGTGGTTTTATTACAGGATCTTTACTGC
AGCCTCAGTGTTTGTCTGCTTTCAGCACACCAAGTTTGGGTTGTAAACT
GTTAATTCTTGGCGAAGATGGAATCCCTATACCACAAAACGCTCCTGGA
ATTGGTGAATTGGCTCTGAATCCCCTCATGTTTGGGGCATCGAGCACAC
TACTAAATGCAAACCACTATGATGTCTACTTTAAAGGCATGCCCTCTTG
GAATGGTAAGGTTCTAAGAAGGCATGGAGATGTATTTGAGCGCACGTCT
AAAGGATACTATCGTGCCCATGGTCGTGCAGATGATACTATGAATCTTG
GGGGTATTAAGGTAAGTTCGGTTGAGATTGAACGTGTATGCAACTCGAT
TGATGACAGAATTCTCGAGACAGCGGCTATAGGGGTTACACCTTCTGGT
GGCGGGCCAGAGAGGTTGGTAATTGTTGTTGCTTTTAAAGATGGCAGTG
GTTCGAAACCCGACTTAATCAAGTTGAAGGTCACACTGAATTCAGCTTT
ACAAAAGAATCTGAACCCTTTGTTTAAGGTTTCTGATGTGGTGCCCTTT
CCATCACTTCCTAGGACAGCAACAAACAAGGTAATGAGAAGGGTTTTGC
GACAGCAGTTGACTCAAATTGGTCAAAATAGCAAGCTATAA.

In some embodiments, the polynucleotide comprises a nucleic acid sequence with at least 89%, at least 92%, at least 95%, or at least 97% homology or identity to SEQ ID NO: 10, or any value and range therebetween. Each possibility represents a separate embodiment of the invention. In some embodiments, the polynucleotide comprises a nucleic acid sequence with 89% to 100%, 92% to 100%, 95% to 100%, or 97% to 100% homology or identity to SEQ ID NO: 10. Each possibility represents a separate embodiment of the invention.

In some embodiments, the polynucleotide comprises or consists of the nucleic acid sequence:

(SEQ ID NO: 11)
ATGAATATAACATTTTCTGTGCCTCCAAAACGCATATTAGTTGATGACC
TGTCTAAAGAAAGCCAGTTATTGCATCCAGGTGGTCGATGGCTTCCCGG
AGCTTATGTAAATCCAGCTAGAAATTGTTTGAGTTTAAGTAGCAAGAGA
AGGTTAAGTGATATAGCAGTTATATGGCGTGATGAAGGAAATGATGATA
TGCCGGTCAACAAAATGACGTTTCAGCAGTTGCGCTCAGAGGTTTGGTT
AGTTGCATATGCACTTGATACATTGGGAGTGGAAAAAGGATCTGCAATT
GCAATCGATATGCCTATGGATGTCAAATCTGTGGTGATTTATCTAGCCA
TTGTTTTAGCAGGCTATGTGGTTGTATCTATTGCAGATAGTTTTGCTGC
TGGTGAAATTTCGACCAGACTTGTATTATCAAAAGCAAAAGCAATTTTT
ACTCAGGATTTGATCATTCGTGGTGACAGAAGCCATCCCTTGTACAGCC
GAGTTGTTGATGCTCAATCACCTCTAGCAATTGTCATTCCTACGAGAGG
CTCAAGTTTTAGTATAAAATTACGTGACGGTGATATTTCTTGGCATGAT
TTTCTGGAACGAGCTAACACTTACAGGAATGTTGAGTTTGTTGCTGTTG
AACGACCCGTTGAAGCTTTCTCAAATATCCTTTTCTCATCAGGAACTAC
AGGGGAACCGAAGGCAATTCCATGGACCCTTGCAACACCTTTCAAGGCT
GGTGCAGACGCTTGGTGCCACATGGATGTCCACAAAGGTGATGTTGTTG
CATGGCCTACTAATCTTGGATGGATGATGGGTCCTTGGCTAATATATGC
TTCATTGTTAAATGGGGGCTCACTTGCATTATACAACGGATCTCCCCTG
ACTTCTGGATTTGCCAAGTTTGTTCAGGATGCAAAAGTAACATTGTTGG
GAGTGATACCAAGTATTGTGAGGGCATGGAGAACAAACAATAGTACAGC
CGGCTTTGACTGGTCAACCATCCGGTGCTTTGGATCGACCGGTGAGGCC
TCTAATACTGATGAATGTCTTTGGCTGATGGGAAGAGCTCATTACAAAC
CGGTCATCGAGTATTGCGGTGGCACAGAGATTGGTGGTGGTTTTATTAC
AGGATCTTTACTGCAGCCTCAGTGTTTGTCTGCTTTCAGCACACCAAGT
TTGGGTTGTAAACTGTTAATTCTTGGCGAAGATGGAATCCCTATACCAC
AAAACGCTCCTGGAATTGGTGAATTGGCTCTGAATCCCCTCATGTTTGG
GGCATCGAGCACACTACTAAATGCAAACCACTATGATGTCTACTTTAAA
GGCATGCCCTCTTGGAATGGTAAGGTTCTAAGAAGGCATGGAGATGTAT
TTGAGCGCACGTCTAAAGGATACTATCGTGCCCATGGTCGTGCAGATGA
TACTATGAATCTTGGGGGTATTAAGGTAAGTTCGGTTGAGATTGAACGT
GTATGCAACTCGATTGATGACAGAATTCTCGAGACAGCGGCTATAGGGG
TTACACCTTCTGGTGGCGGGCCAGAGAGGTTGGTAATTGTTGTTGCTTT
TAAAGATGGCAGTGGTTCGAAACCCGACTTAATCAAGTTGAAGGTCACA
CTGAATTCAGCTTTACAAAAGAATCTGAACCCTTTGTTTAAGGTTTCTG
ATGTGGTGCCCTTTCCATCACTTCCTAGGACAGCAACAAACAAGGTAAT
GAGAAGGGTTTTGCGACAGCAGTTGACTCAAATTGGTCAAAATAGCAAG
CTATAA.

In some embodiments, the polynucleotide comprises a nucleic acid sequence with at least 89%, at least 92%, at least 95%, or at least 97% homology or identity to SEQ ID NO: 11, or any value and range therebetween. Each possibility represents a separate embodiment of the invention. In some embodiments, the polynucleotide comprises a nucleic acid sequence with 89% to 100%, 90% to 100%, 95% to 100%, or 97% to 100% homology or identity to SEQ ID NO: 11. Each possibility represents a separate embodiment of the invention.

In some embodiments, the polynucleotide of the invention comprises 1,200 to 2,500 nucleotides. In some embodiments, the polynucleotide of the invention is 1,200 to 1,500 nucleotides long.

In some embodiments, 1,200 to 2,500 nucleotides comprises: at least 1,250 nucleotides, at least 1,500 nucleotides, at least 1,750 nucleotides, at least 1,950 nucleotides, at least 2,050 nucleotides, at least 2,150 nucleotides, at least 2,250 nucleotides, at least 2,300 nucleotides, or at least 2,450 nucleotides, or any value and range therebetween. Each possibility represents a separate embodiment of the invention. In some embodiments, 1,200 to 2,500 nucleotides comprises: 1,200 to 1,950 nucleotides, 1,300 to 2,250 nucleotides, 1,250 to 2,500 nucleotides, 1,350 to 2,350 nucleotides, 1,550 to 2,200 nucleotides, 1,400 to 2,050 nucleotides, or 1,275 to 2,325 nucleotides. Each possibility represents a separate embodiment of the invention.

In some embodiments, the polynucleotide comprises a plurality of polynucleotides. In some embodiments, the polynucleotide comprises a plurality of types of polynucleotides. As used herein, the term “plurality” comprises any integer equal to or greater than 2. In some embodiments, the polynucleotide comprises at least 2, at least 3, at least 4, at least 5, at least 6, at least 7, at least 8, at least 9, at least 10, or 11 different nucleic acid sequences, or any value and range therebetween, wherein each of the different nucleic acid sequences is selected from SEQ ID Nos.: 1-11. Each possibility represents a separate embodiment of the invention. In some embodiments, the polynucleotide comprises 2-3, 2-4, 2-5, 2-6, 2-7, 2-8, 2-9, 2-10, 2-11, 3-5, 3-7, 3-9, 3-11, 4-5, 4-7, 4-9, 4-11, 5-7, 5-9, 5-11, 6-7, 6-9, 6-11, 7-9, 7-11, 8-9, 8-11, 9-11, or 10-11 different nucleic acid sequences, wherein each of the different nucleic acid sequences is selected from SEQ ID Nos.: 1-11.

In some embodiments, the polynucleotide is a plurality of polynucleotide molecules, wherein each of the plurality of the polynucleotide molecules comprises a different nucleic acid sequence, and wherein the different nucleic acid sequences are selected from SEQ ID Nos.: 1-11.

In some embodiments, the polynucleotide encodes a protein characterized by acyl activating enzymatic (AAE) activity. In some embodiments, the polynucleotide encodes an AAE protein. In some embodiments, the AAE is an AAE derived from Helichrysum umbraculigerum.

As used herein, the terms “acyl activating enzyme” and “AAE” are interchangeable, and refer to any peptide, polypeptide, or a protein, capable of catalyzing the activation of a carboxylic acid. In some embodiments, AAE activity comprises forming or formation of a thioester bond. In some embodiments, AAE activity comprises coupling a carboxyl group to an amine group. In some embodiments, AAE activity comprises coupling a carboxyl group to an alcohol. In some embodiments, the AAE is an acid-thiol ligase.

According to some embodiments, there is provided an artificial nucleic acid molecule comprising the polynucleotide disclosed herein.

In some embodiments, the artificial vector comprises a plasmid. In some embodiments, the artificial vector comprises or is an Agrobacterium comprising the artificial nucleic acid molecule. In some embodiments, the artificial vector is an expression vector. In some embodiments, the artificial vector is a plant expression vector. In some embodiments, the artificial vector is for use in expressing an AAE encoding nucleic acid sequence as disclosed herein. In some embodiments, the artificial vector is for use in heterologous expression of an AAE encoding nucleic acid sequence as disclosed herein in a cell, a tissue, or an organism. In some embodiments, the artificial vector is for use in producing or the production of an acyl-coenzyme A (acyl-CoA) in a cell, a tissue, or an organism.

Expressing of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3: 1671-1680 (1984); and Brogli et al., Science 224:838-843 (1984)] or heat shock promoters, e.g., soybean hspl7.5-E or hspl7.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, Agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide encoding a protein comprising an amino acid sequence as described herein.

According to some embodiments, there is provided a protein encoded by: (a) the polynucleotide disclosed herein; (b) the artificial vector disclosed herein; or the plasmid or Agrobacterium disclosed herein.

In some embodiments, the protein is encoded by a polynucleotide comprising or consisting of SEQ ID Nos.: 1-11.

In some embodiments, the protein comprises an amino acid sequence with at least 82%, at least 85%, at least 87%, at least 89%, at least 90%, at least 93%, at least 95%, at least 97%, or at least homology or identity to any one of SEQ ID Nos.: 12-22.

In some embodiments, the protein is an isolated protein.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

In some embodiments, the protein comprises or consists of the amino acid sequence:

(SEQ ID NO: 12)
MTSSKKFTVEVEPAIPAKDGKPSAGPVYRSIFAKDGFPAHIDGLDSCWD
IFRLSVEKYPNNRMLGTREFVNGKHGPYVWSTYKQVYDKVIKVGNAIRA
CGVEPGGRCGIYGANCAEWIMSMEACNAHGLYCVPLYDTLGAGAIEFIL
CHAEVTIAFVEEKKIPELLKTFPKAGEFLKTIVSFGKVTPEQREQAENF
GLKIHSWDEFLTLGDDKNFDLPLKEKTDICTIMYTSGTTGDPKGVLISN
NSMATLIAGVNRLLDSAKESLNQHDVYLSFLPLAHIFDRVIEECFINHG
ASIGFWRGDVKLLIEDIGELKPTIFCAVPRVLDRIYSGLQQKISAGGFI
KRNLFNLAYSYKLRNMKGGKTHSEASPLSDKIVFSKVKQGLGGNVRIIL
SGAAPLAPHVEAYLKVVACSHVLQGYGLTETCAGSFVSLPNEMEMLGTV
GPPVPVLDARLESVPEMNYDACSSKPQGEICIRGDVLFSGYYKREDLTK
EVFVDGWFHTGDIGEWQPDGSMKIIDRKKNIFKLSQGEYVAVENLENVY
GNVSDIDTIWIYGNSFEFCLVAVVNPNEPAIKRYAEANNISGDFDSLCE
NPKIKEYILGELARIGKEKKLKGFEFVKAVHLDPVPFDMERDLLTPTFK
KKRPQMLKYYQDVIDNMYKTINKK.

In some embodiments, the protein comprises an amino acid sequence with at least 91%, at least 93%, at least 95%, or at least 97% homology or identity to SEQ ID NO: 12, or any value and range therebetween. Each possibility represents a separate embodiment of the invention. In some embodiments, the protein comprises an amino acid sequence with 91% to 100%, 92% to 100%, 93% to 100%, or 95% to 100% homology or identity to SEQ ID NO: 12. Each possibility represents a separate embodiment of the invention.

In some embodiments, the protein comprises or consists of the amino acid sequence:

(SEQ ID NO: 13)
MDALRKPNSANSSPLTPIGFLERAAVVFANSPSIVYNNLIYTWSDTFHR
CLRLASSISRLAIRKGDVVSVLAPNIPAIYELHFGITMTGAIINTINTR
LDARTISILLCHSESKLVFVDYQLTRLIREAVSLMPDACVPPQLVLIVD
DGHNLSLLSDQFINTYEAMVETGDPGFNWVRPDSDWDPLTLNYTSGTTS
SPKGVVNSHRGSFIVAFDSLLEWHVPKQPIMLWTLPMFHANGWSFVWGM
AAVGGTNVCLRKFDATIIYDTIRNHHVTHMCGAPVVLNMLSEGKPLEHT
VHIMTAGAPPPAAVLLRTESLGFEVTHGFGMTETGGLVVSCSWKKEWNR
LPVTEKARLKARQGVRTLGMTEVDIVDPESGVSVTRDGLTQGELVLRGG
SIMLGYLKDPETTNKSVKNGWFYTGDVAVMHPDGYLEIKDRSKDVIISG
GENISSVEVESILYQHPAINEAAVVGRPDEFWGESPCAFVSLKDDNGKV
AVPTADEIMKFCKGKLPGYMVPKSVVFKKDLPKTSTGKIQKYVLRKLAK
DLGFAVKSRI.

In some embodiments, the protein comprises an amino acid sequence with at least 83%, at least 85%, at least 90%, at least 95%, at least 97%, or at least 99% homology or identity to SEQ ID NO: 13, or any value and range therebetween. Each possibility represents a separate embodiment of the invention. In some embodiments, the protein comprises an amino acid sequence with 83% to 100%, 85% to 100%, 90% to 100%, or 95% to 100% homology or identity to SEQ ID NO: 13. Each possibility represents a separate embodiment of the invention.

In some embodiments, the protein comprises or consists of the amino acid sequence:

(SEQ ID NO: 14)
MTEEEKNKAESMGIKTYAWSDFLHLGSKNPSELQTPKATDICTIMYTSG
TSGDPKGVILTHENATTNIRGVDLFMEQFEDKMTVDDVYISFLPLAHIL
DRMIEEYFFRSGASVGFYHGDINALKEDLAELKPTFLAGVPRVLEKIHE
GVLKGLEEVNPRRRKIFSILYNHKLKYMKAGYKHKYASPLADLLAFRKV
KNRLGGRIRLMVSGGAPLSTEIEEFMRVTSCAFVAQGYGLTETCGLATL
GFPDEMCMIGTVGSPFVYTELRLEEVSDMGYDPLANPPRGEICVKGKTP
FAGYYKNPELTNEVMKDGWFHTGDIGEMQPNGVLKIIDRKKHLIKLSQG
EYIALEYLEKVYCITPILEDIWVYGDSFKSSLVAVAVPNKENAEKWADQ
KGLKVSYSELCTLTQFRDYIQSELKSTAERNKLRGFEHIKAIIVEPRTF
EGDQELLTATMKKRRNKLLNRYKEGIDNLYKNLAANKR.

In some embodiments, the protein comprises an amino acid sequence with at least 86%, at least 88%, at least 90%, at least 95%, or at least 99% homology or identity to SEQ ID NO: 14, or any value and range therebetween. Each possibility represents a separate embodiment of the invention. In some embodiments, the protein comprises an amino acid sequence with 86% to 100%, 89% to 100%, 90% to 100%, or 92% to 100% homology to SEQ ID NO: 14. Each possibility represents a separate embodiment of the invention.

In some embodiments, the protein comprises or consists of the amino acid sequence:

(SEQ ID NO: 15)
MVYKSLNSISISDIVNLGISPETATQLHQKLTEIIQIYGFDAPQTWTQI
STRILHPDLPFCFHQMMYYGCYVDFGPDPPAWSPDPKDAKLTNIGSLLE
RRGKEFLGPSYKDPISSYSALQEFSALNLEVFWKTILDEMNITFSVPPK
RILVDDLSKESQLLHPGGRWLPGAYVNPARNCLSLSSKRRLSDIAVIWR
DEGNDDMPVNKMTFQQLRSEVWLVAYALDTLGVEKGSAIAIDMPMDVKS
VVIYLAIVLAGYVVVSIADSFAAGEISTRLVLSKAKAIFTQDLIIRGDR
SHPLYSRVVDAQSPLAIVIPTRGSSFSIKLRDGDISWHDFLERANTYRN
VEFVAVERPVEAFSNILFSSGTTGEPKAIPWTLATPFKAGADAWCHMDV
HKGDVVAWPTNLGWMMGPWLIYASLLNGGSLALYNGSPLTSGFAKFVQD
AKVTLLGVIPSIVRAWRTNNSTAGFDWSTIRCFGSTGEASNTDECLWLM
GRAHYKPVIEYCGGTEIGGGFITGSLLQPQCLSAFSTPSLGCKLLILGE
DGIPIPQNAPGIGELALNPLMFGASSTLLNANHYDVYFKGMPSWNGKVL
RRHGDVFERTSKGYYRAHGRADDTMNLGGIKVSSVEIERVCNSIDDRIL
ETAAIGVTPSGGGPERLVIVVAFKDGSGSKPDLIKLKVTLNSALQKNLN
PLFKVSDVVPFPSLPRTATNKVMRRVLRQQLTQIGQNSKL.

In some embodiments, the protein comprises an amino acid sequence with at least 86%, at least 88%, at least 90%, at least 95%, or at least 99% homology or identity to SEQ ID NO: 15, or any value and range therebetween. Each possibility represents a separate embodiment of the invention. In some embodiments, the protein comprises an amino acid sequence with 86% to 100%, 89% to 100%, 90% to 100%, or 92% to 100% homology to SEQ ID NO: 15. Each possibility represents a separate embodiment of the invention.

In some embodiments, the protein comprises or consists of the amino acid sequence:

(SEQ ID NO: 16)
MGDSEGSSISTPTTEQVGFLSNIMEDKSYSAAVAIMVAIAVPLVLSSVF
AAKKKVKQRGVPVQVGGEPGFAMRNSRSNKLVDVPWEGARTMAALFEQS
CKKHSQLRFLGTRKLIERSFVSGSDGRKFEKLHLGEYQWETYGQIFERV
CNFASGLIQLGHDPDTRIAIFSDTRAEWLIAFEGCFRQNITVVTIYASL
GDDALIHSLNETKVSTLICDSKLLKKVAAVSSSLKTVENFIYFESDNTE
ALNEIGDWKISSFSEVESLGQKSPVSARLPIKKDVAVIMYTSGSTGLPK
GVMMTHGNVVATAAAVMTVIPNIGTNDVYLAYLPLAHIFELAAETVMVT
AGIPIGYGSALTLTDTSNKIKKGTLGDASILKPTLMAAVPAILDRVRDG
VLKKVEEKGGLTTKIFNIAYKRRLLAVDGSWLGAWGLEKLLWDAIVFKK
IRSVLGGDIRFMLCGGAPLAADTQRFINVCVGAPIGQGYGLTETCAGAA
FSEADDNSVGRVGPPLPCVYIKLVSWDEGGYLTSDKPMPRGEVVVGGYS
VTAGYFNNEEKTNEVYKVDESGMRWFYTGDIGRFHPDGCLEIIDRKKDI
VKLQHGEYISLGKVEAALASSKYVENVMLHADPFHTYCVALVVPARQVI
EQWAQDAGISYQDFAELCDKKETVSEVQQSLTKVAKDAKLDKFETPAKI
KLMPDPWTPESGLVTAALKLKREQLKSKFKDDLDKLYG.

In some embodiments, the protein comprises an amino acid sequence with at least 89%, at least 92%, at least 94%, at least 97%, or at least 99% homology or identity to SEQ ID NO: 16, or any value and range therebetween. Each possibility represents a separate embodiment of the invention. In some embodiments, the protein comprises an amino acid sequence with 89% to 100%, 91% to 100%, 93% to 100%, or 95% to 100% homology to SEQ ID NO: 16. Each possibility represents a separate embodiment of the invention.

In some embodiments, the protein comprises or consists of the amino acid sequence:

(SEQ ID NO: 17)
MSVYTVKVEDSRAASGETPSAGPVYRCIYAKDALMELPPGYESPWDFFS
ESVKRNPKNPALGRRQVIDGKAGGYSWLSYQEAYNSALRIASAIRSRSV
NPGDRCGIYGPNCPEWIISMEACNSNGITYVPLYDTLGANAVEYIINHA
EISLVFVQENKLSAILSCLPNCSSNLKTIVSFGKFSESQKNEAMEHGVD
CFSWEEFSSMGNLEDELPAKNKTDICTIMYTSGTTGEPKGVVLSNRAFM
SEVLSMHELLIETDKPGTEEDTYFSFLPLAHIFDQIMETYFIYSGASIG
FWQGDIRYLIEDLLVLQPTIFCGVPRVYDRIYTGIMAKISTGGAIRKAL
FDFAYNYKLRNLEKGIQQDKSAPLLDKLVFDKIKQGFGGRVRLMLSGAA
PLPKHVEEFLRVTCCTVLSQGYGLTESCGGCFTSIANVYSMIGTVGVPM
TTIEARLESVPEMGYDALSSVPCGEICLRGNTLFSGYHKRDDLTDAVLV
DGWFHTGDIGEWQADGAMKIIDRKKNIFKLSQGEYVAVESIESTYSRCP
LVTSIWVYGNSFESFLVAVVVPDRVAVEEFAAKNNESGDYASLCKNPNV
RKYVLEELNAEAQCNKLRGFEMLKAVHLDPVPFDFERDLITPTFKLKRQ
QLLKYYKDCVEQLYAEAKTSKK.

In some embodiments, the protein comprises an amino acid sequence with at least 93%, at least 94%, at least 95%, at least 97%, or at least 99% homology or identity to SEQ ID NO: 17, or any value and range therebetween. Each possibility represents a separate embodiment of the invention. In some embodiments, the protein comprises an amino acid sequence with 93% to 100%, 95% to 100%, 97% to 100%, or 99% to 100% homology to SEQ ID NO: 17. Each possibility represents a separate embodiment of the invention.

In some embodiments, the protein comprises or consists of the amino acid sequence:

(SEQ ID NO: 18)
METHGPRLLGAAYKDPITSYKQFQKFSVQHLEVYWSLVLEKLSIQFQER
PKCIVDTSDKSKHGGTWLPGSVLNIAECCILSTTETDEKVAIVWRDERC
DNLDVNKMTFKELRQQVMLVANALKLLFSKGDPIAIDMPMTVTAVILYL
AIVYSGFVVVSIADSFAAKEIATRLRVSNAKAIFTQDYIVRGGRRFPLY
SRVIEATQCRAIVVPAIGENVEVILRKQDISWGDFLSGAKQLPSPDYCS
PVYQSIDTLTNILFSSGTTGDPKAIPWTQISPMRCAADGWAHMDIQAGD
VYCWPTNLGWVMGPIVLYSSFLTGATLALYNGSPLGHGFGKFVQDAGVT
ILGTVPSIVKSWKSTRCMEGLDWTKIKAFGSTGEASNVDDDLWLSSKAY
YKPVLECCGGTELASSYVQGNLLQPQAFGALSSASMGTGFVIFDDHGVP
YPDDEPCVGEVGLFPVYMGASDRLLNADHEKIYFKGMPSYKGMQLRRHG
DIIKRTIGGYLVVQGRADDTMNLGGIKTSSIEIERVCEQADGSIMETAA
VSVAPATGGPELLAIFVVLKNGCNTQPQDLKMIFSKAIQKNLNPLFKVS
FVKVVPEFPRTASNKLLRRVLRNQVKEELQTRSKI.

In some embodiments, the protein comprises an amino acid sequence with at least 84%, at least 87%, at least 91%, at least 95%, or at least 99% homology or identity to SEQ ID NO: 18, or any value and range therebetween. Each possibility represents a separate embodiment of the invention. In some embodiments, the protein comprises an amino acid sequence with 84% to 100%, 87% to 100%, 90% to 100%, or 95% to 100% homology to SEQ ID NO: 18. Each possibility represents a separate embodiment of the invention.

In some embodiments, the protein comprises or consists of the amino acid sequence:

(SEQ ID NO: 19)
MEITKSIQELGLQDLLNTGLTPNDAKSLQIEIKHIINSQTTNSNPVELW
RQITSAKLLKPSYPHSLHQLIYYAVYCNYDASIYGPPLYWFPSEIDSKR
SNLGNIMETHGPRLLGAAYKDPITSYKQFQKFSVQHLEVYWSLVLEKLS
IQFQERPKCIVDTSDKSKHGGTWLPGSVLNIAECCILSTSETDDKVAIV
WRDERCDNLDVNKMTFKELRQQVMLVANALKLLFSKGDPIAIDMPMTVT
AVILYLAIVYSGFVVVSIADSFAAKEIATRLRVSNAKAIFTQDYIVRGG
RRFPLYSRVIEATQCRAIVVPAIGENVEVILRKQDISWGDFLSGAKQLP
SPDYCSPVYQSIDTLTNILFSSGTTGDPKAIPWTQISPMRCAADGWAHM
DIQAGDVYCWPTNLGWVMGPIVLYSSFLTGATLALYNGSPLGHGFGKFV
QDAGVTILGTVPSIVKSWKSTRCMEGLDWTKIKAFGSTGEASNVDDDLW
LSSKAYYKPVLECCGGTELASSYVQGNLLQPQAFGALSSASMGTGFVIF
DDHGVPYPDDEPCVGEVGLFPVYMGASDRLLNADHEKIYFKGMPSYKGM
QLRRHGDIIKRTIGGYLVVQGRADDTMNLGGIKTSSIEIERVCEQADGS
IMETAAVSVAPATGGPELLAIFVVLKNGCNTQPQDLKMIFSKAIQKNLN
PLFKVFS.

In some embodiments, the protein comprises an amino acid sequence with at least 82%, at least 87%, at least 91%, at least 95%, or at least 99% homology or identity to SEQ ID NO: 19, or any value and range therebetween. Each possibility represents a separate embodiment of the invention. In some embodiments, the protein comprises an amino acid sequence with 82% to 100%, 85% to 100%, 89% to 100%, or 90% to 100% homology to SEQ ID NO: 19. Each possibility represents a separate embodiment of the invention.

In some embodiments, the protein comprises or consists of the amino acid sequence:

(SEQ ID NO: 20)
MVYKSLNSISISDIVNLGISPETATQLHQKLTEIIQIYGFDAPQTWTQI
STRILHPDLPFCFHQMMYYGCYVDFGPDPPAWSPDPKDAKLTNIGSLLE
RRGKEFLGPSYKDPISSYSALQEFSALNLEVFWKTILDEMNITFSVPPK
RILVDDLSKESQLLHPGGRWLPGAYVNPARNCLSLSSKRRLSDIAVIWR
DEGNDDMPVNKMTFQQLRSEVWLVAYALDTLGVEKGSAIAIDMPMDVKS
VVIYLAIVLAGYVVVSIADSFAAGEISTRLVLSKAKAIFTQDLIIRGDR
SHPLYSRVVDAQSPLAIVIPTRGSSFSIKLRDGDISWHDFLERANTYRN
VEFVAVERPVEAFSNILFSSGTTGEPKAIPWTLATPFKAGADAWCHMDV
HKGDVVAWPTNLGWMMGPWLIYASLLNGGSLALYNGSPLTSGFAKFVQD
AKVTLLGVIPSIVRAWRTNNSTAGFDWSTIRCFGSTGEASNTDECLWLM
GRAHYKPVIEYCGGTEIGGGFITGSLLQPQCLSAFSTPSLGCKLLILGE
DGIPIPQNAPGIGELALNPLMFGASSTLLNANHYDVYFKGMPSWNGKVL
RRHGDVFERTSKGYYRAHGRADDTMNLGGIKVSSVEIERVCNSIDDRIL
ETAAIGVTPSGGGPERLVIVVAFKDGSGSKPDLIKLKVTLNSALQKNLN
PLFKVSDVVPFPSLPRTATNKVMRRVLRQQLTQIGQNSKL.

In some embodiments, the protein comprises an amino acid sequence with at least 86%, at least 88%, at least 90%, at least 95%, or at least 99% homology or identity to SEQ ID NO: 20, or any value and range therebetween. Each possibility represents a separate embodiment of the invention. In some embodiments, the protein comprises an amino acid sequence with 86% to 100%, 89% to 100%, 91% to 100%, or 93% to 100% homology to SEQ ID NO: 20. Each possibility represents a separate embodiment of the invention.

In some embodiments, the protein comprises or consists of the amino acid sequence:

(SEQ ID NO: 21)
MTFQQLRSEVWLVAYALDTLGVEKGSAIAIDMPMDVKSVVIYLAIVLAG
YVVVSIADSFAAGEISTRLVLSKAKAIFTQDLIIRGDRSHPLYSRVVDA
QSPLAIVIPTRGSSFSIKLRDGDISWHDFLERANTYRNVEFVAVERPVE
AFSNILFSSGTTGEPKAIPWTLATPFKAGADAWCHMDVHKGDVVAWPTN
LGWMMGPWLIYASLLNGGSLALYNGSPLTSGFAKFVQDAKVTLLGVIPS
IVRAWRTNNSTAGFDWSTIRCFGSTGEASNTDECLWLMGRAHYKPVIEY
CGGTEIGGGFITGSLLQPQCLSAFSTPSLGCKLLILGEDGIPIPQNAPG
IGELALNPLMFGASSTLLNANHYDVYFKGMPSWNGKVLRRHGDVFERTS
KGYYRAHGRADDTMNLGGIKVSSVEIERVCNSIDDRILETAAIGVTPSG
GGPERLVIVVAFKDGSGSKPDLIKLKVTLNSALQKNLNPLFKVSDVVPF
PSLPRTATNKVMRRVLRQQLTQIGQNSKL.

In some embodiments, the protein comprises an amino acid sequence with at least 89%, at least 92%, at least 94%, at least 97%, or at least 99% homology or identity to SEQ ID NO: 21, or any value and range therebetween. Each possibility represents a separate embodiment of the invention. In some embodiments, the protein comprises an amino acid sequence with 89% to 100%, 91% to 100%, 93% to 100%, or 95% to 100% homology to SEQ ID NO: 21. Each possibility represents a separate embodiment of the invention.

In some embodiments, the protein comprises or consists of the amino acid sequence:

(SEQ ID NO: 22)
MNITFSVPPKRILVDDLSKESQLLHPGGRWLPGAYVNPARNCLSLSSKR
RLSDIAVIWRDEGNDDMPVNKMTFQQLRSEVWLVAYALDTLGVEKGSAI
AIDMPMDVKSVVIYLAIVLAGYVVVSIADSFAAGEISTRLVLSKAKAIF
TQDLIIRGDRSHPLYSRVVDAQSPLAIVIPTRGSSFSIKLRDGDISWHD
FLERANTYRNVEFVAVERPVEAFSNILFSSGTTGEPKAIPWTLATPFKA
GADAWCHMDVHKGDVVAWPTNLGWMMGPWLIYASLLNGGSLALYNGSPL
TSGFAKFVQDAKVTLLGVIPSIVRAWRINNSTAGFDWSTIRCFGSTGEA
SNTDECLWLMGRAHYKPVIEYCGGTEIGGGFITGSLLQPQCLSAFSTPS
LGCKLLILGEDGIPIPQNAPGIGELALNPLMFGASSTLLNANHYDVYFK
GMPSWNGKVLRRHGDVFERTSKGYYRAHGRADDTMNLGGIKVSSVEIER
VCNSIDDRILETAAIGVTPSGGGPERLVIVVAFKDGSGSKPDLIKLKVT
LNSALQKNLNPLFKVSDVVPFPSLPRTATNKVMRRVLRQQLTQIGQNSK
L.

In some embodiments, the protein comprises an amino acid sequence with at least 88%, at least 92%, at least 94%, at least 97%, or at least 99% homology or identity to SEQ ID NO: 22, or any value and range therebetween. Each possibility represents a separate embodiment of the invention. In some embodiments, the protein comprises an amino acid sequence with 88% to 100%, 90% to 100%, 92% to 100%, or 95% to 100% homology to SEQ ID NO: 22. Each possibility represents a separate embodiment of the invention.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

In some embodiments, the protein comprises or is characterized by acyl activating enzymatic activity.

In some embodiments, an acyl is selected from C1-C8 alkyl chain, an alpha-unsaturated phenylalkyl carboxylic acid, an alpha-saturated phenylalkyl carboxylic acid.

In some embodiments, an acyl is a C1 alkyl chain. In some embodiments, an acyl is a C2 alkyl chain. In some embodiments, an acyl is a C3 alkyl chain. In some embodiments, an acyl is a C4 alkyl chain. In some embodiments, an acyl is a C5 alkyl chain. In some embodiments, an acyl is a C6 alkyl chain. In some embodiments, an acyl is a C7 alkyl chain. In some embodiments, an acyl is a C8 alkyl chain.

In some embodiments, a C1-C8 alkyl chain is hexanoic acid. In some embodiments, an acyl is hexanoic acid.

In some embodiments, an alpha-unsaturated phenylalkyl carboxylic acid comprises cinnamic acid or a derivative thereof.

In some embodiments, a cinnamic acid derivative comprises a hydroxylated derivative of cinnamic acid.

In some embodiments, a hydroxylated derivative of cinnamic acid comprises or is coumaric acid.

According to some embodiments, there is provided a transgenic cell comprising: (a) the polynucleotide disclosed herein; (b) the artificial nucleic acid molecule disclosed herein; (c) the plasmid or Agrobacterium disclosed herein; (d) the protein disclosed herein; or any combination thereof.

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell is a unicellular organism, a cell of a multicellular organism, and a cell in a culture.

In some embodiments, a unicellular organism comprises a fungus or a bacterium.

In some embodiments, the fungus is a yeast cell.

In some embodiments, the cell is an insect cell. In some embodiments, the cell comprises an insect cell line.

Types of insect cell lines suitable for transformation and/or heterologous expression are common and would be apparent to one of ordinary skill in the art. Non-limiting examples of such insect cell lines include, but are not limited to, Sf-9 cells, SR+ Schneider cells, S2 cells, and others.

According to some embodiments, there is provided an extract derived from a transgenic cell disclosed herein, or any fraction thereof.

In some embodiments, the extract comprises the polynucleotide of the invention, an isolated DNA molecule as disclosed herein, a protein as disclosed herein, or any combination thereof.

According to some embodiments, there is provided a homogenate, lysate, extract, derived from a transgenic cell disclosed herein, any combination thereof, or any fraction thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar or nonpolar solvent), liquid chromatography mass spectrometry, or others.

According to some embodiments, there is provided a transgenic plant, a transgenic plant tissue or a plant part. In some embodiments, there is provided a transgenic plant, or any portion, seed, tissue or organ thereof, comprising at least one transgenic plant cell of the invention. In some embodiments, the transgenic plant, transgenic plant tissue or plant part, comprises: (a) the polynucleotide disclosed herein; (b) the artificial disclosed herein; (c) the plasmid or Agrobacterium disclosed herein; (d) the protein of the invention; (e) the transgenic cell disclosed herein; or any combination thereof.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

In some embodiments, the transgenic plant, transgenic plant tissue, or plant part is or derived from a Cannabis sativa plant. In some embodiments, the transgenic plant is a C. sativa plant.

In some embodiments, the transgenic plant, transgenic plant tissue, or plant part is or derived from hemp. In some embodiments, C. sativa comprises or is hemp.

According to some embodiments, there is provided a composition comprising any one of the herein disclosed: (a) polynucleotide of the invention (for example, an isolated DNA molecule); (b) artificial vector; (c) plasmid or Agrobacterium; (d) protein of the invention; (e) transgenic cell; (f) extract; (g) transgenic plant tissue or plant part; and (h) any combination of (a) to (g), and an acceptable carrier.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Synthesis

According to some embodiments, there is provided a method for synthesizing acyl coenzyme A (CoA).

According to some embodiments, the method comprises the steps: (a) providing a cell comprising an artificial vector comprising a nucleic acid sequence having at least 70%, at least 75%, at least 80%, at least 85%, at least 89%, at least 92%, at least 95%, or at least 99% homology or identity to any one of SEQ ID Nos.: 1-11 or any combination thereof; and (b) culturing the cell from step (a) such that a protein encoded by the artificial vector is expressed, thereby synthesizing acyl CoA. Each possibility represents a separate embodiment of the invention.

According to some embodiments, the method comprises contacting CoA with an acyl group in the presence of a protein comprising an amino acid sequence with at least 75%, at least 80%, at least 85%, at least 90%, at least 93%, or at least 95% homology or identity to any one of SEQ ID Nos.: 12-22 or any combination thereof, thereby synthesizing acyl CoA. Each possibility represents a separate embodiment of the invention.

According to some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell comprises an artificial vector comprising a nucleic acid sequence having at least 70%, at least 75%, at least 80%, at least 85%, at least 89%, at least 92%, at least 95%, or at least 99% homology or identity to any one of SEQ ID Nos.: 1-11 or any combination thereof, or any value and range therebetween. Each possibility represents a separate embodiment of the invention.

In some embodiments, the transgenic cell or the transfected cell comprises the polynucleotide of the invention or a plurality thereof, as disclosed herein.

In some embodiments, the transgenic cell or the transfected cell comprises the artificial nucleic acid molecule or vector as disclosed herein.

In some embodiments, the cell is a transgenic cell, or a cell transfected with a polynucleotide as disclosed herein.

In some embodiments, the culturing comprises supplementing the cell with an effective amount of an acyl group. In some embodiments, the supplementing is via the growth or culture medium wherein the cell is cultured.

In some embodiments, the acyl group is conjugated to CoA so as to obtain the acyl CoA in the presence of the protein. In some embodiments, conjugated is ligated. In some embodiments, the protein catalyzes the formation of a covalent bond between the acyl group and the CoA, thereby producing or catalyzing the formation of acyl CoA.

In some embodiments, the acyl CoA is selected form: acetyl CoA, butyryl CoA, hexanoyl CoA, octanoyl CoA, cinnamoyl CoA, coumaroyl CoA, or any combination thereof.

In some embodiments, the acyl CoA is or comprises hexanoyl CoA.

In some embodiments, the method further comprises a step preceding step (a), comprising introducing or transfecting the cell with the artificial nucleic acid molecule or vector, disclosed herein.

Method for introducing or transfecting a cell with an artificial nucleic acid molecule or vector are common and would be apparent to one of ordinary skill in the art.

In some embodiments, introducing or transfecting comprises transferring an artificial nucleic acid molecule or vector comprising the polynucleotide disclosed herein into a cell; or modifying the genome of a cell to include the polynucleotide disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

As used herein, the terms “transfecting” and “introducing” are interchangeable.

In some embodiments, the contacting is in a cell-free system.

Types of suitable cell-free systems for synthesizing acyl CoA utilizing any one of: the polynucleotide of the invention or a plurality thereof, as disclosed herein, and the protein of the invention, or a plurality thereof, would be apparent to one of ordinary skill in the art.

In some embodiments, the method further comprises a step preceding step (b), comprising separating the cultured transgenic cell or the cultured transfected cell from the medium.

Method for separating cell from a medium are common and may include, but not limited to, centrifugation, ultracentrifugation, or other, as would be apparent to one of ordinary skill in the art.

According to some embodiments, there is provided an extract of a transgenic cell or a transfected cell obtained according to the herein disclosed method.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof; or (c) any combination of (a) and (b), and an acceptable carrier, as described herein.

In some embodiments, a portion comprises a fraction or a plurality thereof.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Chemicals and Reagents

CBGA, acetic acid, propionic acid, butyric acid, pentanoic acid, hexanoic acid, heptanoic acid, octanoic acid, ±2-methyl butyric acid, phenylalanine, and hexanoic-D₁₁ acid were purchased from Sigma-Aldrich (Rehovot, Israel). Acetic-D₃ acid (D>99%), propionic-D₅ acid (D>99%), butyric-D₅ acid (D>98%), pentanoic-D₉ acid (D>98%), heptanoic-D₅ acid (D>99%), octanoic-D₅ acid (D>99%), iso-butyric-D₇ acid (D>98%), ±2-methyl butyric-D ₉ acid (D>99%), iso-valeric-D₉ acid (D>98%), iso-caproic-D₁₁ acid (D>98%) were purchased from C/D/N isotopes (Quebec, Canada). Phenylalanine-D₅ (D>98%) and phenylalanine-¹³C₉, ¹⁵N₁ (¹³C, ¹⁵N>99%) were synthesized by Cambridge Isotope Laboratories (Andover, MA). HeliCBGA (NP009525) was purchased from Analyticon Discovery GmbH (Potsdam, Germany).

### Feeding Experiments

All feeding solutions were prepared as aqua solutions of 0.5 mg ml⁻¹ of the precursor. The pH of the short- and medium-chain chain fatty acid (FA) solutions were adjusted to be in the range of 5.5-6.0. The phenylalanine feeding experiments was performed on leaves from young mother plants excised by cutting at the proximal side of the pedicel with scissors under water (to avoid air penetration into the pedicel, which may influence the feeding efficiency), leaving attached 1-2 cm of the pedicel. For the FA feeding experiments, 10 cm young cuttings were obtained from mother plants. The lower leaves were removed, leaving 4-5 leaves on each stem, and the stem was peeled to increase the intake of the labeled solutions. Three to four leaves or the young cuttings were immersed into Eppendorf tubes with 1.8 ml of the aqua solutions [DDW (control), unlabeled or labeled precursors, each group consisted of a minimum of three biological replicates]. All feeding experiments were performed in a controlled environment for 48-96 h under 25° C. and constant fluorescent illumination and humidity. The tubes were periodically refilled with the specific solutions throughout the experiment. Upon termination, the fresh leaves were rinsed with a small amount of water to remove all traces of externally fed precursor, dried gently, frozen, and ground to a fine powder using mortar and pestle. Next, 100 mg of the frozen powdered plant tissue were extracted with 300 μl ethanol, sonicated for 15 min, agitated in an orbital shaker for 30 min, and then centrifuged at 14,000 g for 10 min. The supernatants were filtered through a 0.22 μm syringe filter, and the samples were analyzed without further dilution.

UPLC-qTOF Analysis of Cannabinoids from H. umbraculigerum Tissues

Fresh samples of six different tissues: young leaves, old leaves, florets and receptacle of flowers, stem, and root were collected from a plant at the flowering stage. Florets and the receptacle of flowers were detached using a scalpel and extracted separately. All the tissues were flash-frozen in liquid N₂, ground in a mortar to a fine powder, and extracted as previously described with 1 ml ethanol.

Samples were analyzed using a high-resolution ultrahigh-performance liquid chromatography-tandem quadrupole time-of-flight (UPLC-qTOF) system comprised of a UPLC (Waters Acquity) with a diode array detector connected either to a XEVO G2-S QTof (Waters) or to Synapt HDMS (Waters). The chromatographic separation of compounds was performed on a 100 mm×21 mm i.d. (internal diameter), 1.7 μm UPLC BEH C18 column (Waters Acquity), The mobile phase consisted of 0.1% formic acid in acetonitrile:water (5:95, v/v; phase A) and 0.1% formic acid in acetonitrile (phase B). The flow rate was 0.3 ml min⁻¹, and the column temperature was kept at 35° C. Compounds were analyzed using a 29 min multistep gradient method: initial conditions were 40% B for 1 min, raised to 100% B until 23 min, held at 100% B for 3.8 min, decreased to 40% B until 27 min, and held at 40% B until 29 min for re-equilibration of the system. Electrospray ionization (ESI) was used in negative ionization with an m/z range of 50-1,000 Da. Masses of the eluted compounds were detected with the following settings: capillary 1 kV, source temperature 140° C., desolvation temperature 450° C., and desolvation gas flow 800 1 h⁻¹. Argon was used as the collision gas. MS/MS experiments were performed in negative ionization mode according to the observed deprotonated masses. The following settings were used: a capillary spray of 1 kV; cone voltage of 30 eV; collision energy ramp of 15-50 eV.

### Trichome Isolation

Young leaves were harvested and soaked in ice-cold, distilled water and then abraded using a BeadBeater machine (Biospec Products, Bartlesville, OK). The polycarbonate chamber was filled with 15 g of plant material, and with half the volume with glass beads (0.5 mm diameter), XAD-4 resin (1 g/g plant material), and ethanol 80% to full volume. Leaves were beaten by 2-4 pulses of operation of 1 min each. This procedure was carried out at 4° C., and after each pulse the chamber was allowed to cool on ice. Following abrasion, the contents of the chamber were first filtered through a kitchen mesh strainer and then through a 100 μm nylon mesh to remove the plant material, glass beads, and XAD-4 resin. The residual plant material and beads were scraped from the mesh and rinsed twice with additional ethanol 80% that was also passed through the 100 μm mesh. The presence of enriched glandular trichome secretory cells was checked by visualization in an inverted optical microscope.

Genome Sequencing and Assembly of H. umbraculigerum

The genome size of H. umbraculigerum was estimated by flow cytometry. Briefly, nuclei were isolated by chopping young leaf tissue of Helichrysum and tomato (used as known reference) in isolation buffer. The samples were stained with propidium iodide, and at least 10,000 nuclei were analyzed in a flow cytometer, and the ratio of G1 peak means between both samples was calculated. High molecular weight DNA was extracted from young frozen leaves and sent for sequencing in the Genome Center of UC Davis. The DNA quality was checked by TapeStation traces and a Qubit fluorimeter (Thermo Fisher). Sequencing was done in a Pacbio Sequel II platform, and a ˜12-kilobase DNA SMRT bell library was prepared according to the manufacturer's protocol. Three different SMRT 8M cells were used, yielding 57.8 Gb of HiFi data (˜44×haploid coverage). In addition to Pacbio HiFi data, 200 M reads of PE 2×150 Illumina Hi-C data were obtained by Phase Genomics. Hifiasm software was used to integrate both Pacbio HiFi and HiC data to produce chromosome-scale and haplotype-resolved assemblies.

Further scaffolding of the primary assembly was performed using the Hi-C data and the SALSA software. Ragta g was used for a final round of ordering using the primary assembly as reference to reach syntenic scaffolds for each haplotype. Visualizations of Hi-C data were performed with Juicer and whole-genome alignments with the pafr package (https://dwinter.github.io/pafr/). Finally, the assembly was softmasked for repetitive elements using EDTA.

RNA Sequencing and Genome Annotation of H. umbraculigerum

RNA was extracted from seven different tissues: young leaves, old leaves, florets and receptacles of flowers, stems, roots and trichomes. RNA integrity was checked using a TapeStation instrument. Paired-end Illumina libraries were prepared for five of the tissues and sequenced on Illumina HiSeq 3000 instrument (PE 2×150, ˜40 M reads per sample). Random sequencing errors were corrected using Rcorrector18, and uncorrectable reads were removed. Adaptor and quality trimming were performed using TrimGalore! with the following parameters: —length 36 —q 5 —stringency 1-e 0.1 (https://github.com/FelixKrueger/TrimGalore). Ribosomal RNA was filtered by discarding reads mapping to SILVA_132_LSURef and SILVA_138_SSURef non-redundant databases using bowtie2—very-sensitive-local mode. Fastq quality checks on each of the steps were performed using MultiQC. The remaining reads were pooled and used for genome-guided de novo transcriptome assembly using Trinity. The Iso-Seq data were obtained from four of the tissues and processed using isoseq3 and cDNA Cupcake ToFU pipelines (https://github.com/Magdoll/cDNA_Cupcake). Fused and unspliced transcripts were removed, and only polyA positive transcripts were kept for a unique set of high-quality isoforms. Iso-Seq and Trinity transcripts were aligned to the assembly using minimap2 and the BAM files were used in the PASA pipeline to generate RNA-based gene model structures. In addition, the novo gene structures were obtained using the software braker2 and the mentioned BAM files as extrinsic training evidence. Finally, ab initio and RNA-based gene models were combined using EvidenceModeler and a final round of PASA pipeline. Gene functional annotation was performed for the predicted mature transcripts using TransDecoder (https://github.com/TransDecoder/TransDecoder), which considers HMMER hits against PFAM and BLASTP hits against UniProt databases for similarity retention criteria. Further annotation of protein-coding transcripts was performed by BLASTP searches against curated plant protein databases and GO and KEGG terms were obtained with Triannotate.

UMI-based 3′ RNAseq of three replicates of the seven tissues was obtained similarly as described. Adaptor and quality trimming were performed using TrimGalore! in two steps, including PolyA trimming mode. Reads were mapped to the genome using STAR, UMI-deduplicated using umitools, and counts were obtained with featureCounts. Normalization was performed with the varianceStabilizingTransformation algorithm of DESeq2, and the CEMItools package was used for coexpression analysis (dissimilarity threshold of 0.6, pvalue of 0.1). Genes in modules with expression profiles in concordance with the presence of the metabolites of interest were analyzed. Candidate genes were selected based on functional annotations, and blast hits with known enzymes.

AAE Expression in E. coli BL21 (DE3) Cells and Protein Purification

All AAE genes from H. umbraculigerum were individually cloned into the pET28b vector and expressed in E. coli BL21 (DE3) cells. Bacterial cells were grown in LB medium at 37° C. When cultures reached Absorbance 600=0.6, protein expression was induced with 200 μM of isopropyl-1-thio-β-d-galactopyranoside (IPTG) at 15° C., for 24 h. Bacterial cells were lysed by sonication in 50 mM Tris-HCl pH 7.5, 500 mM NaCl, 1 mM PMSF, 10% glycerol and protease inhibitor cocktail (Sigma Aldrich). An aliquot of whole-cell extract was kept for further analysis. Soluble protein was purified on Ni-NTA agarose beads (Adar Biotech) and eluted with 300 mM imidazole in buffer containing 50 mM NaH₂PO₄ pH-7.5 and 300 mM NaCl. The whole-cell extract, and the eluted fractions were analyzed by SDS-PAGE stained with InstatBlue.

### AAE Enzyme Assay

Acyl-CoA synthetase assays were performed in a 20 μl reaction mix that contained 0.1 μg recombinant AAE, 50 mM HEPES pH 9, 8 mM ATP, 10 mM MgCl2, 0.5 mM CoA and 4 mM sodium hexanoate for 10 min at 40° C. Reactions were terminated with 2 μl of 1 M HCl and stored on ice until analysis. After centrifugation at 15 000 g for 5 min at 4° C., the samples were diluted 1:100 in water and analyzed by UPLC-MS/MS (Stout et al., 2012). injections were performed on a UPLC (Waters) connected to a Triple Quad detector (TQ-S, Waters) in multiple reaction monitoring (MRM) mode. The chromatographic separation was achieved using a similar column as previously described. The mobile phase consisted of an aqueous buffer pH 7.0 (10 mM Ammonium Acetate, 5 mM NH₄HCO₂, phase A) and acetonitrile (phase B). The flow rate was 0.3 ml min⁻¹, and the column temperature was kept at 25° C. Compounds were analyzed using a 15 min multistep gradient method: initial conditions were 1% B raised to 35% B until 10.5 min, and then raised to 100% B until 11 min, held at 100% B for 1 min, decreased to 1% B until 12.5 min, and held at 1% B until 15 min for re-equilibration of the system. The instrument was operated in positive mode with a capillary voltage of 3.0 kV, and a cone voltage of 50 V. Metabolite identity was confirmed with authentic standards (Sigma Aldrich). Two different transitions were used for analysis of: acetyl-CoA (810.52>303.30, 27.0V; 810.52>428.25, 24.0V); butyryl-CoA (838.58>331.30, 28.0 V; 838.58>331.30, 25.0 V); hexanoyl-CoA (866.65>359.40, 28.0 V; 866.65 >428.25, 26.0 V); octanoyl-CoA (894.65>387.55, 30.0 V; 894.65>428.25, 28.0 V); coumaroyl-CoA (914.59>407.37, 30.0 V; 914.59>428.25, 28.0 V); cinnamoyl-CoA (898.59>391.37, 30.0 V; 898.59>428.25, 28.0 V).

### EXAMPLE 1

### UPLC-qTOF Profiling of Helichrysum Tissues

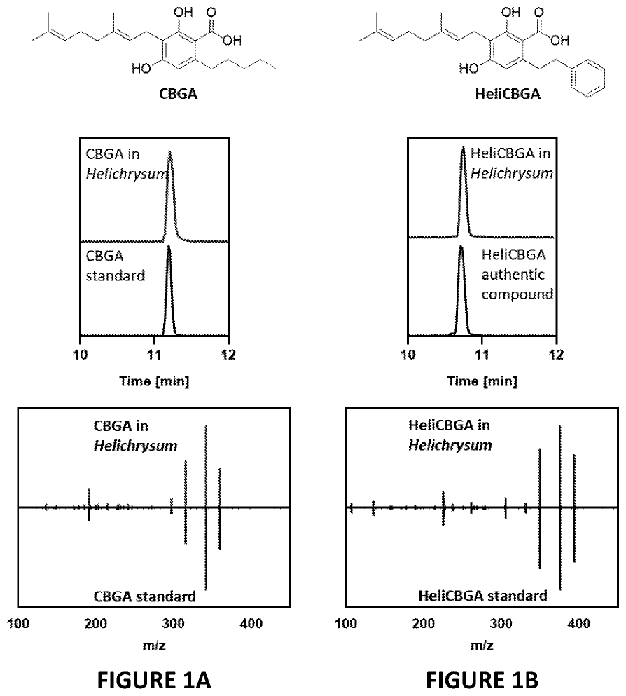

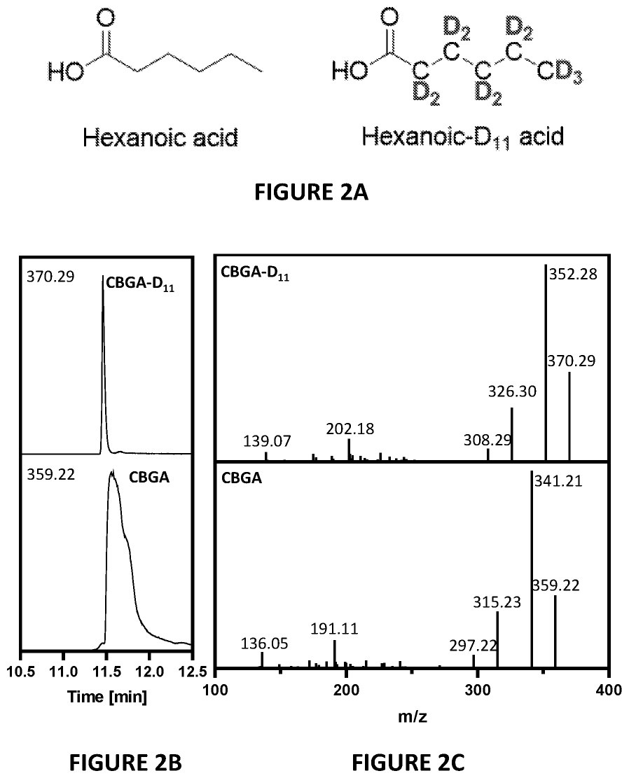

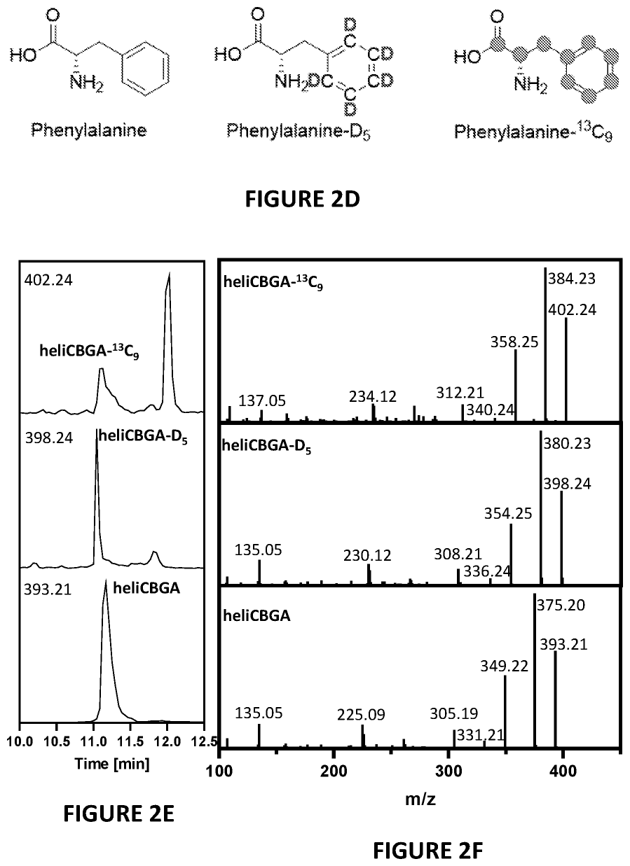

Six tissues (young leaf, old leaf, florets and receptacles of flowers, stems, and roots) of H. umbraculigerum were first profiled using UPLC--qTOF. CBGA and its phenethyl analog (heliCBGA,) were observed in all the tissues besides roots. These compounds were identified by comparison to analytical standards (FIGS. 1A-1B). While hexanoyl CoA is the committed precursor for alkyl type cannabinoids, the precursor for aralkyl type cannabinoids is not yet identified. Here, it was demonstrated via feeding of isotopically labeled hexanoic acid (hexanoic-D₁₁ acid) and phenylalanine (phenylalanine-D₅ or phenylalanine-¹³C₉), that these compounds are precursors of CBGA and heliCBGA, respectively (FIG. 2).

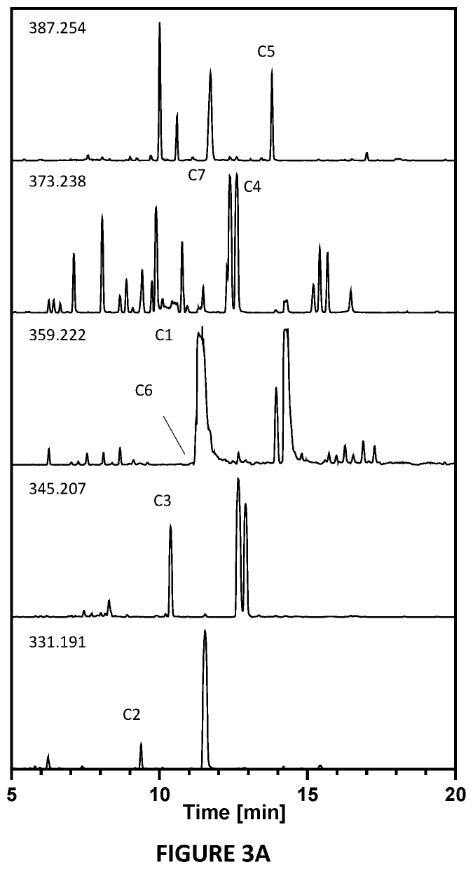

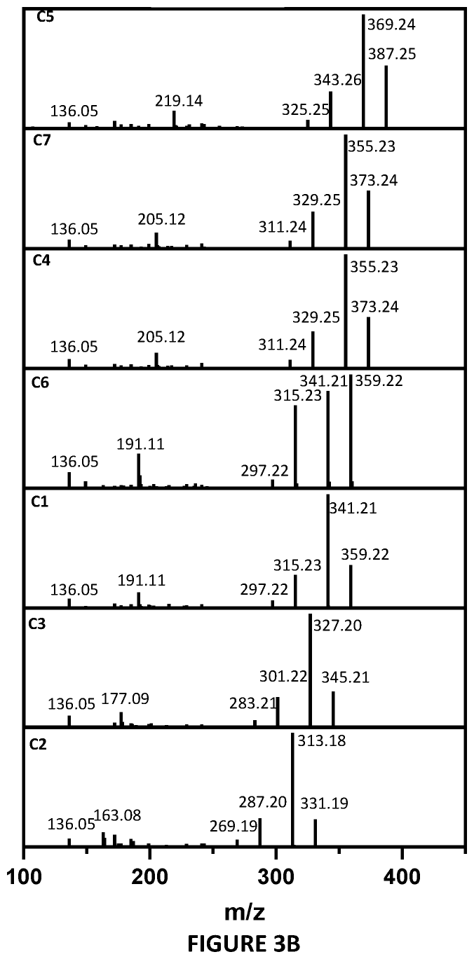

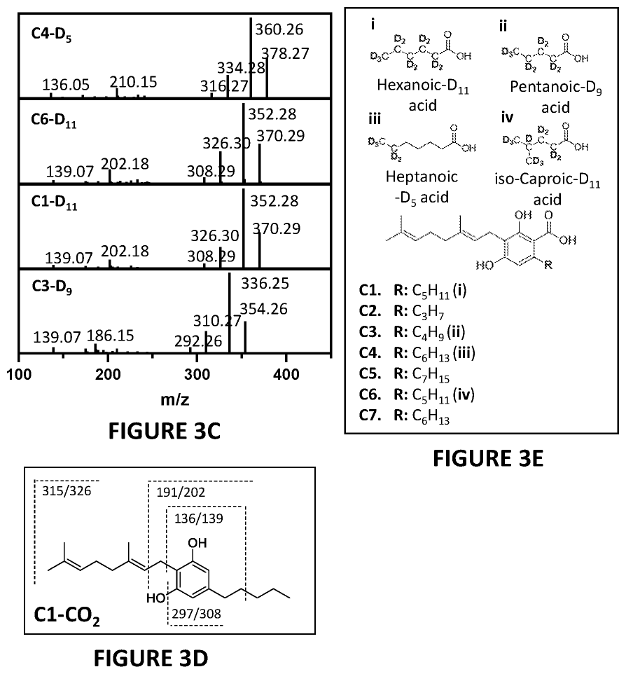

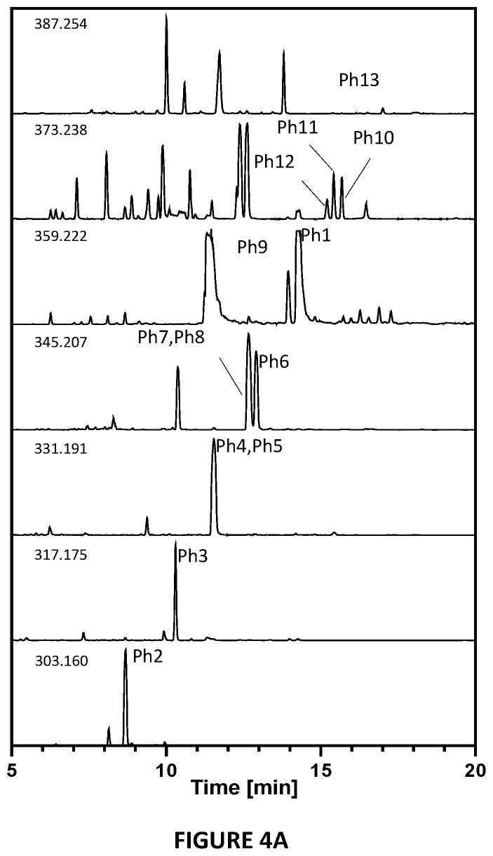

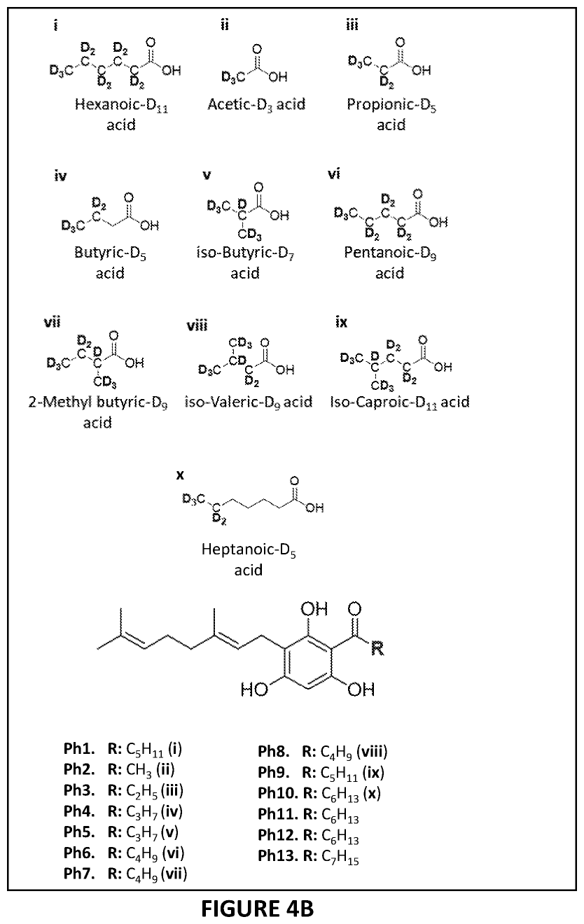

Cannabis also produces other CBGA-type analogs with aliphatic chains of varying lengths (one to seven carbons), which derive from different linear short-chain FAs. Several of these analogs were also observed in H. umbraculigerum for the first time including, cannabigerovarinic acid (CBGVA, C2, FIG. 3), cannabigerol butyric acid (CBGBA, C3, FIG. 3), cannabigerohexolic acid (CBGHA, C4, FIG. 3), and cannabigerophorolic acid (CBGPA, C5, FIG. 3), corresponding to three, four, six, and seven carbon chains, respectively. The identification of compounds was via feeding of isotopically labeled short- and medium-chain fatty acids (FAs) with varying chain lengths, and via MS/MS fragmentation spectra. Two compounds that eluted before CBGA and CBGHA with similar masses and fragmentation patterns were identified as branched cannabinoids (C6,and C7, FIG. 3). These branched cannabinoids are not identified in Cannabis. Additional prenylated acyl phloroglucinoids derived from similar precursors as the cannabinoids, according to the feeding experiments (FIG. 4).

### EXAMPLE 2

### Functional Characterization of Acyl Activating Enzymes (AAEs)

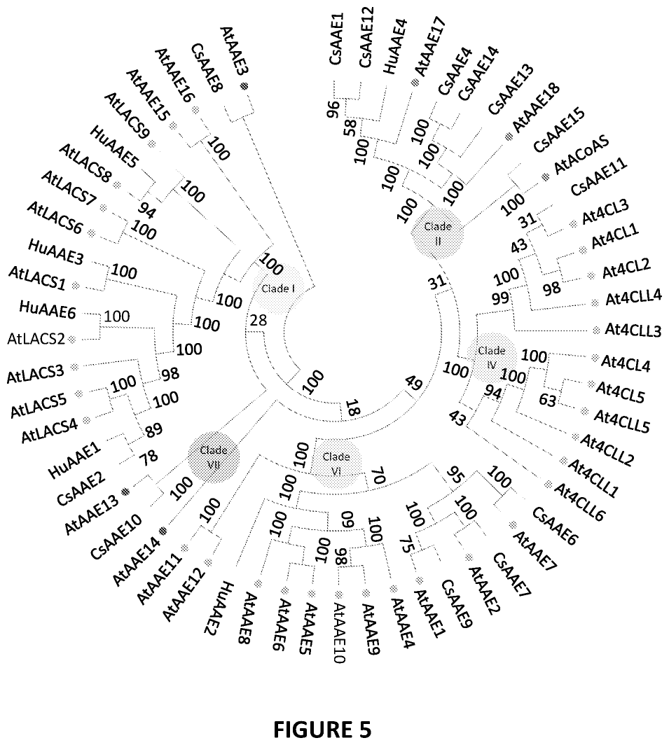

In Cannabis, the hexanoyl coenzyme A (CoA) precursor for cannabinoid biosynthesis is formed by trichome specific acyl activating enzyme 1 (CsAAE1) (Stout et al., 2012). CsAAE1 activates hexanoate and other short- and medium-chain fatty acids to form their corresponding CoAs. Based on the H. umbraculigerum transcriptome analysis, the inventors identified eleven candidate genes that encode putative AAEs. Among them, HuAAE4 (SEQ ID NO: 4) from H. umbraculigerum showed great homology to CsAAE1 (˜68% at amino acid level, FIG. 5).

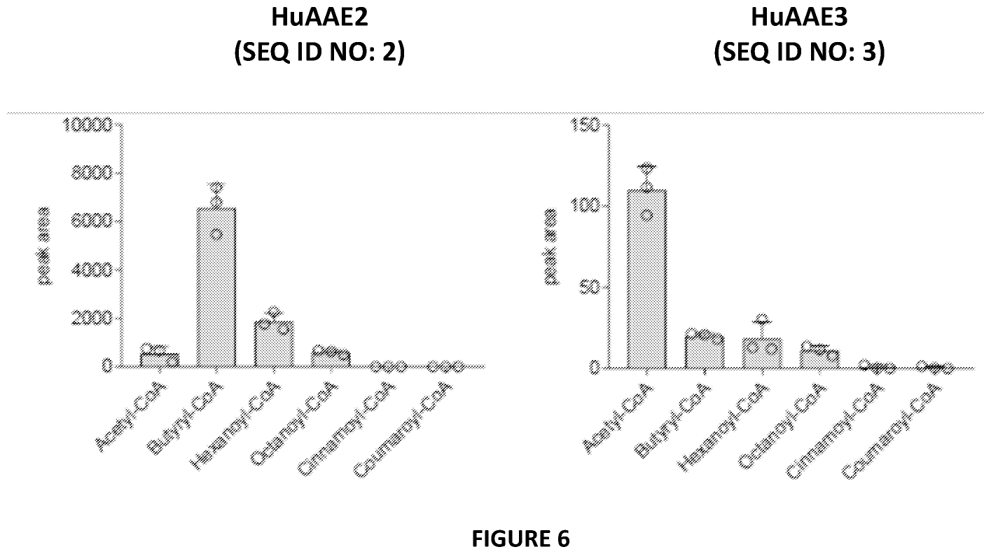

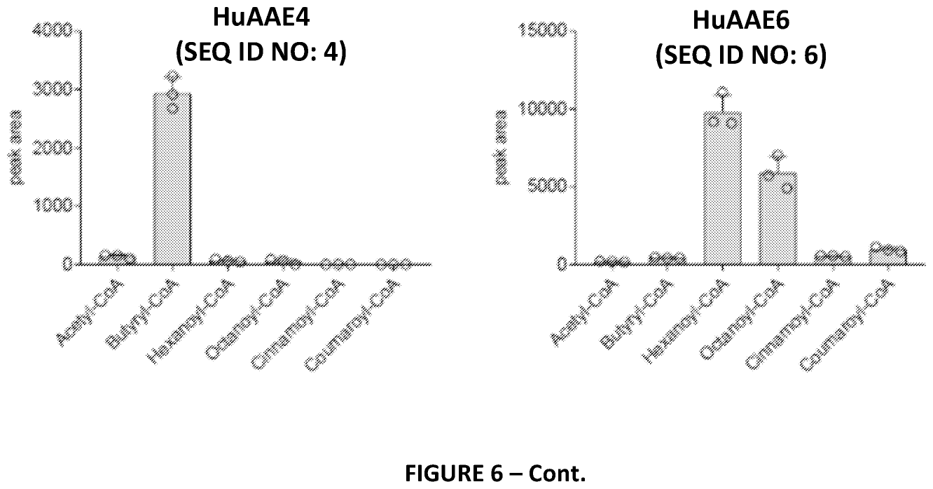

Next, the inventors individually recombinantly expressed six of the eleven AAEs, each in E. coli, purified respective proteins, and examined their activity using array of substrates (acetic acid, butyric acid, hexanoic acid, octanoic acid, cinnamic acid and coumaric acid). While HuAAE2 (SEQ ID NO: 2) and HuAAE4 (SEQ ID NO: 4) enzymes efficiently produced butyryl CoA compared to other substrates, HuAAE3 (SEQ ID NO: 3) showed great activity against acetic acid and formed acetyl CoA (FIG. 6). These AAE enzymes showed negligible or low activity towards cinnamic acid and coumaric acid substrates. Interestingly, HuAAE6 (SEQ ID NO: 6) showed very high activity towards medium chain fatty acids like hexanoic acid and octanoic acid, yet it also reacted with cinnamic acid and coumaric acid to form cinnamoyl CoA and coumaroyl CoA, respectively (FIG. 6). Thus, HuAAE6 was shown to activate both alkyl (e.g., hexanoic acid and octanoic acid) and aralkyl (e.g., cinnamic acid and coumaric acid) precursors required for alkyl- and aralkyl type-cannabinoids biosynthesis in H. umbraculigerum.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. An isolated DNA molecule comprising a nucleic acid sequence having at least 89% homology to SEQ ID Nos.: 1-11, or any combination thereof.

2. The isolated DNA molecule of claim 1, wherein said nucleic acid sequence having at least 89% homology to any one of SEQ ID Nos.: 1-11 is 1,200 to 2,500 nucleotides long.

3. The isolated DNA molecule of claim 1, wherein said nucleic acid sequence encodes a protein characterized by acyl activating enzymatic activity.

4. An artificial nucleic acid molecule comprising the isolated DNA molecule of claim 1 or a nucleic acid sequence having at least 89% homology thereto, or any combination thereof.

5. A plasmid or an Agrobacterium comprising the isolated DNA molecule of claim 1 or a nucleic acid sequence having at least 89% homology thereto, or any combination thereof.

6. An isolated protein encoded by the isolated DNA molecule of claim 1.

7. The isolated protein of claim 6, comprising an amino acid sequence with at least 93% homology to any one of SEQ ID Nos.: 12-22 or consisting thereof.

8. (canceled)

9. The isolated protein of claim 6, being characterized by acyl activating enzymatic activity, wherein said acyl is selected from the group consisting of: C1-C8 alkyl chain, and alpha-unsaturated phenylalkyl carboxylic acid, and optionally wherein any one of said C1-C8 alkyl chain is hexanoic acid, said alpha-unsaturated phenylalkyl carboxylic acid comprises cinnamic acid or a derivative thereof, said cinnamic acid derivative is a hydroxylated derivative of cinnamic acid, said hydroxylated derivative of cinnamic acid is coumaric acid, and any combination thereof.

10.-14. (canceled)

15. A transgenic cell comprising the isolated DNA molecule of claim 1, or a nucleic acid sequence having at least 89% homology thereto, wherein said transgenic cell being any one of: a unicellular organism, a cell of a multicellular organism, and a cell in a culture, and optionally wherein said unicellular organism comprises a fungus or a bacterium, said fungus is a yeast cell, or both.

16.-18. (canceled)

19. An extract derived from the transgenic cell of claim 15, or any fraction thereof, and optionally wherein said extract comprises an isolated protein encoded from said isolated DNA molecule.

20. (canceled)

21. A transgenic plant, a transgenic plant tissue or a plant part, comprising the isolated DNA molecule of claim 1 or a nucleic acid sequence having at least 89% homology thereto, or any combination thereof, and optionally wherein said transgenic plant being a Cannabis sativa plant

22. (canceled)

23. A composition comprising, the isolated DNA molecule of claim 1, and an acceptable carrier.

24. A method for synthesizing acyl coenzyme A (CoA) comprising the steps:
a. providing a cell comprising an artificial vector comprising a nucleic acid sequence having at least 89% homology to any one of SEQ ID Nos.: 1-11; and
b. culturing said cell from step (a) such that a protein encoded by said artificial vector is expressed,
thereby synthesizing acyl CoA.

25. The method of claim 24, wherein any one of: (i) said protein is characterized by having an acyl activating enzymatic activity; (ii) said culturing comprises supplementing said cell with an effective amount of an acyl group, and optionally wherein said acyl group is conjugated to said CoA so as to obtain said acyl CoA in the presence of said protein; (iii) said acyl group is selected from the group consisting of: C1-C8 alkyl chain, and alpha-unsaturated phenylalkyl carboxylic acid, and optionally wherein said C1-C8 alkyl chain is hexanoic acid, said cinnamic acid derivative is a hydroxylated derivative of cinnamic acid, said hydroxylated derivative of cinnamic acid is coumaric acid, or any combination thereof; (iv) said artificial vector is an expression vector; (v) said cell is a prokaryote cell or a eukaryote cell; (vi) said cell is a transgenic cell or a cell transfected with an isolated DNA molecule comprising a nucleic acid sequence having at least 89% homology to SEQ ID Nos.: 1-11, or any combination thereof; (vii) said acyl CoA is selected form the group consisting of: acetyl CoA, butyryl CoA, hexanoyl CoA, octanoyl CoA, cinnamoyl CoA, coumaroyl CoA, and any combination thereof; and (viii) any combination of (i) to (vii), and optionally wherein said method further comprises a step preceding step (a), comprising introducing or transfecting said cell with an artificial vector comprising an isolated DNA molecule comprising a nucleic acid sequence having at least 89% homology to SEQ ID Nos.: 1-11, or any combination thereof.

26.-36. (canceled)

37. A method for synthesizing acyl CoA comprising contacting CoA with an acyl group in the presence of a protein comprising an amino acid sequence with at least 93% homology to any one of SEQ ID Nos.: 12-22, thereby synthesizing acyl CoA.

38. The method of claim 37, wherein any one of: (i) said acyl group is selected from the group consisting of: C1-C8 alkyl chain, and alpha-unsaturated phenylalkyl carboxylic acid; (ii) said C1-C8 alkyl chain is hexanoic acid; (iii) said cinnamic acid derivative is a hydroxylated derivative of cinnamic acid; (iv) said hydroxylated derivative of cinnamic acid is coumaric acid; (v) said acyl CoA is selected form the group consisting of: acetyl CoA, butyryl CoA, hexanoyl CoA, octanoyl CoA, cinnamoyl CoA, coumaroyl CoA, and any combination thereof; (vi) said contacting is in a cell-free system; and (vii) any combination of (i) to (vi).

39.-43. (canceled)

44. A method for obtaining an extract from a transgenic cell comprising the steps:
a. culturing the transgenic cell of claim 15 in a medium; and
b. extracting said transgenic cell,
thereby obtaining an extract from the transgenic cell, and optionally wherein said method further comprises a step preceding step (b), comprising separating said cultured transgenic cell from said medium.

45. (canceled)

46. An extract of a transgenic cell obtained according to the method of claim 44.

47. A medium or a portion thereof separated from a cultured transgenic cell, obtained according to the method of claim 44.

48. A composition comprising the extract of claim 46, and an acceptable carrier.
